(19) United States
(12) Patent Application Publication
(10) Pub. No.: US 2024/0047033 A1
(43) Pub. Date: Feb. 8, 2024

(54) MULTI-OMICS METHODS FOR PRECISION MEDICINE

(71) Applicant: UNIVERSITY OF PITTSBURGH-OF THE COMMONWEALTH SYSTEM OF HIGHER EDUCATION, PITTSBURGH, PA (US)

(72) Inventor: XIAOSONG WANG, SEWICKLEY, PA (US)

(73) Assignee: UNIVERSITY OF PITTSBURGH-OF THE COMMONWEALTH SYSTEM OF HIGHER EDUCATION, PITTSBURGH, PA (US)

(21) Appl. No.: 18/257,349

(22) PCT Filed: Dec. 16, 2021
(86) PCT No.: PCT/US2021/072950

§ 371 Date: Jun. 14, 2023

Related U.S. Application Data
(60) Provisional application No. 63/126,682, filed on Dec. 17, 2020.

(51) Int. Cl. G16H 20/10; G01N 33/574

(52) U.S. Cl. CPC G16H 20/10; G01N 33/57484; G01N 2800/52

(57) ABSTRACT

Provided is a method of modelling a therapeutic response of a cancer cell or tumor, comprising: calculating a weight for each of a plurality of high-dimensional redundant multi-omics features that predict agent sensitivity or other clinical features based on statistical or machine learning methods; and calculating an integral genomic signature score for the cancer cell or tumor based on the weights, while algorithmically resolving the feature redundancy based on unlabeled genomic datasets for large cohorts of human tumors. Also provided is an iGenSig model for an agent that calculates the probability of response or resistance of a patient having a cancer or tumor to treatment with the agent. Also provided is a method for selecting a patient having a cancer or tumor for treatment with an agent. Further provided is a method of treating a patient having a cancer or tumor.

### CROSS-REFERENCE TO RELATED APPLICATIONS

The instant application is entitled to priority under 35 U.S.C. § 119(e) to U.S. Provisional Application No. 63/126,682, filed Dec. 17, 2020, which is hereby incorporated by reference in its entirety.

### GOVERNMENT CONTRACT

This invention was made with government support under grant numbers P30 CA047904-31, CA181368, CA183976 and CA237964 awarded by the National Institutes of Health (NIH). The government has certain rights in the invention.

### FIELD

The general inventive concepts relate to precision medicine (e.g., in oncology), and, in particular, to an integral genomic signature (iGenSig) analysis method for use in big data-based precision medicine, such a precision oncology and immuno-oncology.

### BACKGROUND

Multi-omics is a biological analysis approach in which the data sets that are used are multiple “omics” data, such as the genome, proteome, transcriptome, epigenome, metabolome, and microbiome. Precision oncology, defined as molecular profiling of tumors to achieve customized patient care, has entered the mainstream of cancer patient care¹. The current standard practices for precision oncology include detecting actionable mutations via genetic testing (i.e., EGFR mutation, ALK rearrangements), or detecting small-sized predictive or prognostic gene signatures via targeted expression assays (i.e., Oncotype DX, MammaPrint). Such assays, however, require at least one assay per decision, which limit their cost-effectiveness. On the other hand, the past ten years have observed stunning reduction of sequencing cost for a human genome from $300,000 to $1000, with $100 whole genome sequencing expected soon². With this rate, it is expected that transcriptome and genome sequencing will become the clinical routine for patients. With the advent of low-cost genome sequencing, precision oncology is at the cusp of a deep transformation via leveraging the big data to provide a wide array of clinical decision supports which is deemed to be cost-effective. On the other hand, the computational approaches that can leverage these big data to facilitate clinical decisions and provide tailored health care are far lacking. For example, in metastatic lung cancer, the target therapies prescribed based on the current modeling of genomic sequencing data produced only minimal gain of quality-adjusted life year³. Innovative and robust clinical big data-based decision support models for precision oncology will be of vital importance.

In recent years, there has been great enthusiasm about the potential of artificial-intelligence based clinical decision support systems for big data based precision medicine, however, to date only few examples exist that impact clinical practice⁴. The main challenge is that, multi-OMIC big data typically contain daunting amounts of high-dimensional features but limited number of subjects which pose great challenges to the computational power and training process of artificial intelligence (AI)-based methods. In addition, AI approaches are “black box” tools, so that the algorithmic and biological mechanisms underlying the models are largely unknown. The modeling process is controlled by AI which make it difficult to interpret complex model predictions and is often plagued with the problems of overfitting and overweighing. In addition, there is a lack of big-data based methods specifically addressing the insufficient performance of the prediction models for crossing dataset modeling resulting from the common biases in detected genomic features across different datasets arising from sequencing errors, different library preparation methods and platforms, discordant sequencing depth and read-length, heterogenous sample qualities, and experimental variations etc. This calls for robust, transparent, and explainable methods that can predict clinical treatment outcome from multi-OMIC data with substantially improved resilience against sequencing biases.

There remains a need for multi-omics methods for precision medicine.

### SUMMARY

Provided is a method of modelling a therapeutic response of a cancer cell or tumor, comprising: calculating a weight for each of a plurality of redundant multi-omics features that predict agent sensitivity or other clinical features based on statistical or machine learning methods; and calculating a genomic signature score for the cancer cell or tumor based on the weights.

In some embodiments, the method includes reducing the effect of feature redundancy via adaptively penalizing the redundant features detected in specific samples based on co-occurrence assessed using large cohorts of human cancer cells, cell lines or tumors.

In some embodiments, the cell lines are Genomics of Drug Sensitivity in Cancer Project (GDSC) and/or Cancer Cell Line Encyclopedia (CCLE) cell lines.

In some embodiments, the genomic signature score of a given cancer cell or tumor is calculated using the below formula or its modifications:

$${GenSig}_{\vert Cell {Line}_{x}} = \frac{{\sum }_{i = 1}^{n} {EW}_{i}}{{EFN}_{i}} = \frac{{\sum }_{i = 1}^{n} {I}_{\{ i ∈ x \}} \frac{{ω}_{i}}{{ε}_{i}}}{\frac{n}{{\overline{ε}}_{T}}}$$

wherein ε is a penalization factor, ω is a weight, and EW is Effective Weight.

In some embodiments, the weights are calculated based on weighted Kolmogorov-Smirnov (K-S) tests of Act Area or Area Under the Curve (AUC).

In some embodiments, the method is implemented by a computer.

Provided is an iGenSig model for an agent that calculates the probability of response of a patient having a cancer or tumor to treatment with the agent.

In some embodiments, the model is generated according to the method of any one of the previous embodiments.

In some embodiments, the agent is an EGFR inhibitor, a HER2 inhibitor, a CDK 4/6 inhibitor, a HDAC inhibitor, a BCL2 inhibitor, a JAK inhibitor, a PARP inhibitor, a ERK inhibitor, a MEK inhibitor, a BRAF inhibitor, irinotecan, topotecan, paclitaxel, 5-FU, Vincristine, Venetoclax, Epirubicin, or combinations thereof.

In further embodiments, the EGFR inhibitor is erlotinib, lapatinib, Afatinib, AZD3759. In further embodiments, the BRAF inhibitor is encorafenib, vemurafenib, or dabrafenib. In further embodiments, the MEK inhibitor is binimetinib, cobimetinib, selumetinib, or trametinib. In further embodiments, the HER2 inhibitor is neratinib, trastuzumab, dacomitinib, lapatinib, tucatinib, or pertuzumab. In further embodiments, the CDK4/6 inhibitor is Ribociclib. In further embodiments, the HDAC inhibitor is CAY10603, AR-42, In further embodiments, the BCL2 inhibitor is Venetoclax, in further embodiments, the PARP inhibitor is Niraparib. In further embodiments, the ERK inhibitor is ERK_6604. In yet further embodiments, the JAK inhibitor is AZ960.

Also provided is a method for selecting a patient having a cancer or tumor for treatment with an agent, said method comprising: employing an iGenSig model for the agent to calculate the probability of response of the patient to treatment with the agent; and selecting the patient for treatment with the agent if the probability of response is above a chosen threshold of sensitive iGenSig score and/or de-implementing the treatment with the agent to a patient if the probability of resistance is above a chosen threshold of resistant iGenSig score. The probability of response is linked to a high sensitive iGenSig score for the respective drug or combination. The probability of resistance is linked to a high resistant iGenSig score for the respective drug or combination.

In some embodiments, the method further comprises administering the agent to the patient. In some embodiments, an effective amount of the agent is administered to the patient.

In some embodiments, at least one step of the method is implemented by a computer.

In some embodiments, the agent comprises a pharmaceutically acceptable carrier.

In some embodiments, the chosen threshold is a probability of 50-95% predicted response rate, or 50-95% predicted non-response or resistance rate.

In some embodiments, the iGenSig model is generated according to the method of any one of the embodiments described herein.

In some embodiments, the agent is an EGFR inhibitor, a HER2 inhibitor, a CDK 4/6 inhibitor, a HDAC inhibitor, a BCL2 inhibitor, a JAK inhibitor, a PARP inhibitor, a ERK inhibitor, a MEK inhibitor, a BRAF inhibitor, irinotecan, topotecan, paclitaxel, 5-FU, Vincristine, Venetoclax, Epirubicin, or combinations thereof.

In further embodiments, the EGFR inhibitor is erlotinib, lapatinib, Afatinib, AZD3759. In further embodiments, the BRAF inhibitor is encorafenib, vemurafenib, or dabrafenib. In further embodiments, the MEK inhibitor is binimetinib, cobimetinib, selumetinib, or trametinib. In further embodiments, the HER2 inhibitor is neratinib, trastuzumab, dacomitinib, lapatinib, tucatinib, or pertuzumab. In further embodiments, the CDK4/6 inhibitor is Ribociclib. In further embodiments, the HDAC inhibitor is CAY10603, AR-42, In further embodiments, the BCL2 inhibitor is Venetoclax, in further embodiments, the PARP inhibitor is Niraparib. In further embodiments, the ERK inhibitor is ERK 6604. In yet further embodiments, the JAK inhibitor is AZ960.

### DESCRIPTION OF THE FIGURES

FIGS. 1A-1B shows the principle, workflow, and algorithms of the integral genomic signature modeling approach. FIG. 1A shows the workflow and algorithms of integral genomic signature analysis. The upper panel shows the calculation of the weights for significant genomic features that predict drug sensitivity or resistance based on weighted K-S tests of Act Area or AUC for each drug respectively, and the lower panel shows the computation of a similarity matrix for genomic features based on TCGA Pan-Cancer dataset to penalize the redundancy between the genomic features associated with each cell line x. The resulting sensitive or resistant genomic signature scores are calculated using the indicated formula based on the K-S tests using Act Area or AUC respectively. The dot plot shows the sensitive and resistant iGenSig scores for all cell line subjects, with red and blue colors indicating sensitive and resistant cell lines. FIG. 1B shows a schematic showing the principle and key features of iGenSig modeling: i) the iGenSig approach intentionally retains and creates redundant genomic features, a concept like the use of redundant steel rods to reinforce the pillars of a building. ii) iGenSig modeling utilizes the average correlation intensities of significant genomic features detected in specific samples to diminish the effect of false positive detection resulting from sequencing errors and prevent overweighing. iii) iGenSig modeling extract the second genomic information from unlabeled genomic datasets for large cohorts of human cancers/tumors, in addition to the labeled genomic datasets of drug sensitivity, which will substantially improve its cross-dataset applicability, particularly on clinical trial datasets. iv) iGenSig modeling is a white box approach, thus will be more interpretable and controllable than machine learning or deep learning approaches.

FIGS. 2A-2D show the performance of iGenSig models in predicting the drug responses of GDSC cell lines. FIG. 2A: The performance of the iGenSig models for 369 drugs assessed by their average AUROC. The drugs with top performing models (AUROC >0.85) are shown as barchart on the right. The average AUROC for each drug was calculated based on five train/test sets. FIG. 2B: Correlating the performance of the iGenSig models for 369 drugs with their average number of significant genomic features. The drug models assessed on the five clinical trial datasets are highlighted in red. FIG. 2C shows the performance of the iGenSig model for Lapatinib in predicting the response of GDSC cell lines based on a representative training and testing set. Left, sensitive and resistant GenSig scores for GDSC cell lines. Middle, the correlation of the iGenSig scores with AUC measurements for Lapatinib. Right, the receiver operating characteristic (ROC) curve for predicting sensitive responses to Lapatinib. As golden standard for the ROC curve, the cell line subjects in the test set are divided into sensitive and non-sensitive groups based on the AUC measurements for Lapatinib using the cutoff determined by the waterfall method (see Examples). FIG. 2D: shows clustering GDSC cancer cell lines and targeted kinase drugs based on iGenSig scores. The drugs targeting different kinases or different kinase families form distinctive clusters.

FIGS. 3A-3C show predictive values of iGenSig models developed from GDSC pharmacogenomic dataset on the sensitivity of CCLE profiled cell lines to the respective drugs. FIG. 3A shows the performance of GDSC iGenSig models in predicting the responses of GDSC testing cell lines and CCLE cell lines to 14 drugs shared between the two datasets. 80% of GDSC cell lines are used for building the iGenSig models and 20% of GDSC cell lines are used for testing. 100% of CCLE cell lines are used for cross-dataset validations. If the same drug is profiled by both GDSC batch 1 and 2, the drug sensitivity data from the batch 1 are used in the analysis. FIG. 3B: The predictive values of the iGenSig models developed from GDSC data on the CCLE cell lines treated with Irrinotecan, Nilotinib, Lapatinib, or Erlotinib. Upper panel shows the correlation between the iGenSig scores and the Act Areas of the respective drugs for CCLE cell lines. The horizontal dashed lines shows the cut offs for sensitive (red) and resistant (blue) calls. The vertical dashed line shows the optimal cut off for iGenSig scores determined based on AUROC. The lower panel shows the ROC curves of iGenSig scores in determining the sensitive cell lines vs non-sensitive cell lines. FIG. 3C shows that GDSC and CCLE cell lines show consistent integral genomic signature that correlates with Erlotinib responses. The significant genomic features (n=8540) based on K-S tests are shown in the figure. The GDSC and CCLE cell lines are first sorted by their sensitive iGenSig scores; the cell lines with sensitive iGenSig scores less than the median are then sorted by the resistant iGenSig cores. The cell lines that have been tested for Erlotinib chemical perturbations are shown in the figure, and the sensitive and resistant cell line subjects are indicated as yellow and blue bars.

FIGS. 4A-4D show predictive values of the iGenSig model for Erlotinib developed from GDSC cell line pharmacogenomic data on the survival of patient subjects from the U.S. BATTLE trial and Swiss SAKK 19/05 trial. FIG. 4A: Left, Kaplan-Meier plot showing the predictive values of GDSC iGenSig model for Erlotinib on the patients from the U.S. BATTLE trial. A data-driven cut point of high iGenSig scores was determined as previously described¹. The P-value is based on log-rank test. Right, the differences of iGenSig scores among patients that achieved (Y) or did not achieve (N) 8-week disease control in the Erlotinib, Sorafenib, and Vandetinib treatment arms. Patients with EGFR or KRAS mutations are depicted with red or blue colors. The p-values are based on student's t-test. FIG. 4B: the predictive values of the GDSC iGenSig model for Erlotinib on the patient subjects from the Swiss SAKK 19/05 trial. Left, the ROC curve showing the performance of sensitive iGenSig scores on predicting the objective responses of patient subjects at 12 weeks following Erlotinib and Avastin treatment in the Swiss SAKK 19/05 clinical trial. Right, the predictive value of iGenSig model for Erlotinib does not depend on EGFR mutation status. The p-values are based on student's t-test. FIG. 4C: The network of upregulated and downregulated pathways characteristic of Erolotinib sensitive GDSC cell line subjects. The top upregulated and downregulated pathways clustered in the respective interconnected networks are shown in the figure. The CSEA enrichment score for each pathway in the Erlotinib sensitive signature is depicted by the size of each node. The pathway associations are depicted by the thickness of the edge. The pathway associations are calculated based on CSEA association scores between each pair of pathway. FIG. 4D: Heatmap showing the associations of EMT markers and master transcription factors, as well as ZEB1 and MYC target genes with the sensitive iGenSig scores for Erlotinib in the GDSC, BATTLE, and SAKK10/05 clinical trial datasets. The cell lines and patient subjects are sorted based on their sensitive iGenSig scores.

FIGS. 5A-5E show predictive values of the iGenSig model for 5-FU developed from GDSC cell line pharmacogenomic data on the survival of patient subjects from the French CIT multi-center postsurgical colon cancer patient cohort. FIG. 5A: shows a Kaplan-Meier plot showing the predictive values of the GDSC iGenSig model for 5-FU on the patients from the French CIT cohort treated with S-FU monotherapy. A data-driven cut point of high iGenSig scores was determined as previously described¹ and the P-value is calculated based on log-rank test. FIG. 5B: shows the predictive values of the GDSC iGenSig model for 5-FU on the overall survival of all patient subjects from the CIT cohort treated with different adjuvant chemotherapy regiments or untreated. The BRAF and KRAS mutation status for each subject are indicated by colored dots. The p-values are based on student's t-test. FIG. 5C: shows the predictive values of the GDSC iGenSig models for irinotecan and S-FU on the patient subjects treated with FOLFIRI regimen in the CIT study. The patients are stratified based on their overall survival and their sensitive iGenSig scores based on the two models are plotted. FIG. SD: shows the network of upregulated and downregulated pathways characteristic of 5-FU sensitive GDSC cell line subjects. The top upregulated and downregulated pathways clustered in the respective interconnected networks are shown in the figure. The CSEA enrichment score for each pathway in the Erlotinib sensitive signature is depicted by the size of each node. The pathway associations are depicted by the thickness of the edge. The pathway associations are calculated based on CSEA association scores between each pair of pathway. FIG. 5E: shows a heatmap showing the associations of EMT markers and master transcription factors, ZEB1 and MYC target genes, and interferon γ responsive genes with the sensitive iGenSig scores for 5-FU in the GDSC and CIT datasets. The cell lines and patient subjects are sorted based on their sensitive iGenSig scores for 5-FU.

FIGS. 6A-6B show the predictive of the GDSC iGenSig models on the survival of patient subjects from the multicenter breast cancer patient cohort treated with neoadjuvant taxane-anthracycline chemotherapy. FIG. 6A: Kaplan-Meier plot showing the predictive values of the GDSC iGenSig model for Paclitaxel on the distant recurrence free survival of patients with basal-like TNBC treated with neoadjuvant taxane-anthracycline chemotherapy. A data-driven cut point of high iGenSig scores was determined as previously described¹ and the P-value is determined based on log-rank test. FIG. 6B: The predictive value of the GDSC iGenSig model for paclitaxel on the pathological responses of the enrolled basal-like TNBC patient subjects stratified based on tumor stages. The source cohorts of patient subjects are indicated by colored dots. The p-values annotated on panel a, b, and d are based on student's t-tests.

FIGS. 7A-7D shows the Prediction performance of the iGenSig algorithm and machine learning methods on the U.S. BATTLE trial (FIG. 7A), and Swiss SAKK 19/05 trial (FIG. 7B), French CIT colon cancer clinical study (FIG. 7C), and neoadjuvant taxane-anthracycline study (FIG. 7D). The predictive models were generated based on 80% cell lines from GDSC with five permutated training sets. For AI methods, the unsupervised learning was performed by autoencoder (AE) and supervised learning was performed using various machine learning tools including elastic net (EN), random forest (RF) and support vector machine (SVM). The p-values are based on student's t-tests.

FIG. 8 shows a schematic showing the key difference between integral genomic signature modeling and conventional gene signature or AI methods in handling high-dimensional features. An integral genomic signature is defined as the comprehensive set of high-dimensional genomic features predictive of a given clinical phenotype such as therapeutic response. iGenSig represents a new class of modeling methods that directly utilize high-dimensional genomic signature for predictive modeling based on multi-omics data.

FIGS. 9A-9B show the prediction performance of iGenSig models generated based on genomic features devoid of primary drug targets, or based on solid cancer cell lines. FIG. 9A: the box plot shows the performance of the iGenSig models for Erlotinib, Lapatinib, and Sorafenib assessed on GDSC testing set (left) or CCLE validation set (right) in the presence or absence of the genomic features derived from the respective drug targets. FIG. 9B: The violin plot shows the performance of the GDSC iGenSig models for 14 drugs on the CCLE solid cancer cell lines. The GDSC iGenSig models are generated based on either Pan-cancer cell lines or solid cancer cell lines.

FIG. 10. shows a Kaplan-Meier plot showing the predictive value of the GDSC iGenSig models for Erlotinib on the PDX subjects treated with Erlotinib monotherapy. The P-value is calculated based on log-rank test.

FIG. 11 shows the expression of upregulated gene signature in EMT in association with the iGenSig scores for Erlotinib in GDSC cell lines and patient subjects from the BATTLE trial. The cell line and patient subjects are sorted decreasingly by their iGenSig scores.

FIG. 12 is a heatmap showing the associations of EMT markers and master transcription factors, ZEB1 and MYC target genes, and interferon γ responsive genes with the sensitive iGenSig scores for 5-FU in the GDSC and CIT subjects classified based on cancer types. The cell lines and patient subjects are sorted based on their cancer types and sensitive iGenSig scores for 5-FU.

FIG. 13 is a schematic showing the workflow of deep learning and machine learning methods implemented in this study for drug sensitivity prediction.

### DETAILED DESCRIPTION

While the general inventive concepts are susceptible of embodiment in many forms, there are shown in the drawings, and will be described herein in detail, specific embodiments thereof with the understanding that the present disclosure is to be considered an exemplification of the principles of the general inventive concepts. Accordingly, the general inventive concepts are not intended to be limited to the specific embodiments illustrated herein.

It is also to be understood that the terminology used herein is for the purpose of describing particular embodiments only, and is not intended to be limiting.

The articles “a” and “an” are used herein to refer to one or more than one (i.e., to at least one) of the grammatical object of the article. By way of example, “a cell” means one cell or more than one cell.

“About” as used herein when referring to a measurable value such as an amount, a temporal duration, and the like, is meant to encompass variations of ±5%, preferably ±1%, and still more preferably ±0.1% from the specified value, as such variations are appropriate to perform the disclosed methods.

As used herein, the term “number” shall mean one or an integer greater than one (i.e., a plurality).

As used herein, the terms “component” and “system” are intended to refer to a computer related entity, either hardware, a combination of hardware and software, software, or software in execution. For example, a component can be, but is not limited to being, a process running on a processor, a processor, an object, an executable, a thread of execution, a program, and/or a computer. By way of illustration, both an application running on a server and the server can be a component. One or more components can reside within a process and/or thread of execution, and a component can be localized on one computer and/or distributed between two or more computers. While certain ways of displaying information to users are shown and described with respect to certain figures or graphs as screenshots, those skilled in the relevant art will recognize that various other alternatives can be employed.

The term “pharmaceutically acceptable carrier” as used herein means a pharmaceutically acceptable material, composition or vehicle, such as a liquid or solid filler, diluent, excipient, solvent or encapsulating material.

The term “effective amount” of an agent, as used herein, is an amount sufficient to effect beneficial or desired results, including clinical results. An effective amount can be administered in one or more administrations.

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

The disclosed concept will now be described, for purposes of explanation, in connection with numerous specific details in order to provide a thorough understanding of the subject invention. It will be evident, however, that the disclosed concept can be practiced without these specific details without departing from the spirit and scope of this innovation.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination

As described in more detail elsewhere herein, the disclosed concept provides an integral genomic signature (iGenSig) analysis method as a new class of transparent, manageable, interpretable and resilient method for precision oncology based on multiple types of genome-wide sequencing data. Applicant postulates that the redundant high-dimensional genomic features, which are typically eliminated through dimensionality reduction or feature removal during multi-omics modeling, may help overcome the sequencing biases. Thus, the disclosed concept provides a novel design that directly models the therapeutic response using the high-dimensional features predictive of tumor response to therapies or other clinical features, which the disclosed concept terms as an integral genomic signature (iGenSig). The disclosed concept then mathematically resolves the feature redundancy tailored for each patient subject. Using the genomic dataset of chemical perturbations, the disclosed concept developed the iGenSig models for predicting targeted-therapy responses and tested the applicability of selected models to independent pharmacogenomic datasets for cancer cell lines, patient-derived xenografts, and cancer patients in clinical trials. The iGenSig models demonstrated outstanding performance on predicting patient responses in clinical trial datasets compared to machine learning and deep learning methods. Their predictive powers appear to correlate with the abundance of predictive genomic features. In particular, the iGenSig model for the EGFR inhibitor Erlotinib significantly predicted patient responses in two clinical trials, biological interpretation of which led to new insights into the predictive signature pathways with clinical relevance. Together, iGenSig provides a computational infrastructure to empower tailored cancer therapy based on genome-wide sequencing data.

### Methods of Modelling a Therapeutic Response

Provided herein are methods of modelling a therapeutic response of a cancer cell or tumor.

Provided is a method of modelling a therapeutic response of a cancer cell or tumor, comprising: calculating a weight for each of a plurality of redundant multi-omics features that predict agent sensitivity or other clinical features based on statistical or machine learning methods; and calculating an integral genomic signature score for the cancer cell or tumor based on the weights.

In some embodiments, the method includes reducing the effect of feature redundancy via adaptively penalizing the redundant features detected in specific samples based on co-occurrence assessed using large cohorts of human cancer cells, cell lines, or tumors.

In some embodiments, the cell lines are GDSC and/or CCLE cell lines.

In some embodiments, the genomic signature score of a given cancer cell or tumor is calculated using the below formula or its modifications:

$${GenSig}_{\vert Cell {Line}_{x}} = \frac{{\sum }_{i = 1}^{n} {EW}_{i}}{{EFN}_{i}} = \frac{{\sum }_{i = 1}^{n} {I}_{\{ i ∈ x \}} \frac{{ω}_{i}}{{ε}_{i}}}{\frac{n}{{\overline{ε}}_{T}}}$$

wherein ε is a penalization factor, ω is a weight, and EW is Effective Weight.

In some embodiments, the weights are calculated based on weighted Kolmogorov-Smirnov (K-S) tests of Act Area or Area Under the Curve (AUC).

In some embodiments, the method is implemented by a computer.

To define the weight (ωᵢ) of each genomic feature in predicting sensitive drug responses, the weighted Kolmogorov-Smirnov (WKS) statistics are leveraged to test the enrichment of the feature-positive cell line in the cell line panel sorted in descending order based on Act Area. To prevent bias, genomic features defining fewer than 5 cell lines may be excluded during the calculation of GenSig scores.

Weights for each genomic feature are calculated in predicting resistant drug responses based on the cell line panel sorted by AUC in descending order. The significance of the observed enrichment score (ES) is assessed by comparing that to random ES scores calculated by random features with the same numbers of positive cell lines. This step is repeated until a large number random enrichment scores are calculated, for example 1000.

Normalized enrichment score (NES) is calculated by:

NES=ES/mean(ES_{random}) 1)

The p-values are determined based on the chance of random ES scores to be above the observed ES score for feature i, and the false discovery rate (FDR) were calculated using R package “qvalue”. Significant genomic features with a q-value<0.1 were selected for calculating GenSig scores, which however, should be tuned for different drugs. Some of the genes have both upregulation and downregulation features ranked as significant for predicting the sensitive drug response. The genes that have both upregulated features with FDR <0.1, and downregulated features with FDR <0.3, and the genes that have both downregulated features with FDR <0.1, and upregulated features with FDR <0.3 are thus filtered. On the other hand, some of the genes have only level-1 DGE features selected as significant based on FDR <0.1, but none of their corresponding high levels of DGE features have an FDR more than 0.3 even if they define more than ten cell lines. These genomic features represent noises and are thus filtered as well.

To prevent the inflation of iGenSig scores from feature redundancy, the TCGA Pan-Cancer RNA-seq and exome datasets were leveraged to assess the co-occurrence between genomic features associated with each cell line and generated the cosine similarity matrix of genomic features based on Otsuka-Ochiai coefficient between these features (Kᵢⱼ). The cosine similarity matrixes are then clustered based on Ward's method (D2) using the R module “hClust”. The correlated feature groups are then determined based on an adaptive dynamic cluster detection method², using the parameters: dynamic.method=“hybrid”, cutTree.depth=2, and minClusterSize=40. A penalization factor (ε) is calculated for each genomic feature i based on the similarity indices of the colinear genomic features associated with a given cell line ₓ and of the same cluster as feature i:

εᵢ=Σ_{j∈Cluster}_{ᵢ}Kᵢⱼ 2)

Where Kᵢⱼ; the Otsuka-Ochiai coefficient between feature i and a given genomic feature j from the same cluster group as feature i associated with cell line ₓ, To eliminate the cumulative effect of small overlaps between genomic features, the Otsuka-Ochiai coefficients were adjusted to 0 if Kᵢⱼ<0.1. Here e, is an estimator of redundancy among the genomic features associated with cell line ₓ. The penalization factor ranges from 1 (all genotypes are completely different each other) to n (all genotypes are the same). The weight a) was then penalized using et, resulting in Effective Weight (EW):

$${EW}_{i} = \frac{{ω}_{i}}{{ε}_{i}} \qquad 3 )$$

The trimmed mean of εᵢ (trim=0.3) was then used to calculate the Effective Feature Number (EFN):

$${EFN}_{i} = \frac{n}{{\tilde{ε}}_{T}} \qquad 4 )$$

Finally, the GenSig score of the given cell lineₓ is computed as:

$${GenSig}_{\vert Cell {Line}_{x}} = \frac{{\sum }_{i = 1}^{n} {EW}_{i}}{{EFN}_{i}} = \frac{{\sum }_{i = 1}^{n} {I}_{\{ i ∈ x \}} \frac{{ω}_{i}}{{ε}_{i}}}{\frac{n}{{\overline{ε}}_{T}}} \qquad 5 )$$

The sensitive and resistant iGenSig scores are calculated separately based on the significant genomic features selected for predicting sensitive or resistant responses. The sensitive iGenSig scores are used for assessing the performance of the iGenSig models on predicting sensitive cell lines and patient subjects.

iGenSig Models

Provided is an iGenSig model for an agent that calculates the probability of response of a patient having a cancer or tumor to treatment with the agent.

In some embodiments, the model is generated according to any of the methods described herewith.

In some embodiments, the agent is an EGFR inhibitor, a HER2 inhibitor, a CDK 4/6 inhibitor, a HDAC inhibitor, a BCL2 inhibitor, a JAK inhibitor, a PARP inhibitor, a ERK inhibitor, a MEK inhibitor, a BRAF inhibitor, irinotecan, topotecan, paclitaxel, 5-FU, Vincristine, Venetoclax, Epirubicin, or combinations thereof.

In further embodiments, the EGFR inhibitor is erlotinib, lapatinib, Afatinib, AZD3759. In further embodiments, the BRAF inhibitor is encorafenib, vemurafenib, or dabrafenib. In further embodiments, the MEK inhibitor is binimetinib, cobimetinib, selumetinib, or trametinib. In further embodiments, the HER2 inhibitor is neratinib, trastuzumab, dacomitinib, lapatinib, tucatinib, or pertuzumab. In further embodiments, the CDK4/6 inhibitor is Ribociclib. In further embodiments, the HDAC inhibitor is CAY10603, AR-42, in further embodiments, the BCL2 inhibitor is Venetoclax, in further embodiments, the PARP inhibitor is Niraparib. In further embodiments, the ERK inhibitor is ERK_6604. In yet further embodiments, the JAK inhibitor is AZ960.

### Method for Selecting a Patient

Also provided is a method for selecting a patient having a cancer or tumor for treatment with an agent, said method comprising: employing an iGenSig model for the agent to calculate the probability of response of the patient to treatment with the agent; and selecting the patient for treatment with the agent if the probability of response is above a chosen threshold of sensitive iGenSig score and/or de-implementing the treatment with the agent to a patient if the probability of resistance is above a chosen threshold of resistant iGenSig score.

In some embodiments, the chosen threshold is a probability of 50-95% predicted response rate, or 50-95% predicted non-response or resistance rate. In some embodiments, the chosen threshold is a probability of at least 50%1, at least 60%, at least 70%, at least 80%, at least 90% predicted response rate, or at least 50%, at least 60%, at least 70%, at least 80%, at least 90% predicted non-response or resistance rate.

In some embodiments, the iGenSig model is generated according to the method of any one of the preceding embodiments. In some embodiments, the iGenSig model is generated according to the method described herein.

In some embodiments, the method further comprises administering the agent to the patient. In some embodiments, an effective amount of the agent is administered to the patient.

In some embodiments, at least one step of the method is implemented by a computer

In some embodiments, the agent comprises a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil, or injectable organic esters. In some embodiments, when such pharmaceutical compositions are for human administration, particularly for injection or implantation, the aqueous solution is pyrogen-free or substantially pyrogen-free.

Excipients can be chosen, for example, to effect delayed release of an agent or to selectively target one or more cells, tissues or organs.

The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration.

A pharmaceutically acceptable carrier can contain a physiologically acceptable compound or mixture that acts, for example, to stabilize, increase solubility or to increase the absorption of an agent that is to be administered to a patient. The choice of a pharmaceutically acceptable carrier, including a physiologically acceptable compound or mixture, depends, for example, on the route of administration.

In some embodiments, the agent can be administered to the subject by any number of routes of administration including, for example, orally, absorption through the oral mucosa (e.g., sublingually), anally, rectally or vaginally (e.g., as a pessary, cream or foam), parenterally (including intramuscularly, intravenously, subcutaneously or intrathecally as, for example, a sterile solution or suspension), nasally, intraperitoneally, subcutaneously, transdermally (e.g. a patch), and topically (e.g., cream, ointment, spray, eye drop). The agent may also be formulated for inhalation.

In some embodiments, the agent is an EGFR inhibitor, a HER2 inhibitor, a CDK 4/6 inhibitor, a HDAC inhibitor, a BCL2 inhibitor, a JAK inhibitor, a PARP inhibitor, a ERK inhibitor, a MEK inhibitor, a BRAF inhibitor, irinotecan, topotecan, paclitaxel, 5-FU, Vincristine, Venetoclax, Epirubicin, or combinations thereof.

In further embodiments, the EGFR inhibitor is erlotinib, lapatinib, Afatinib, AZD3759. In further embodiments, the BRAF inhibitor is encorafenib, vemurafenib, or dabrafenib. In further embodiments, the MEK inhibitor is binimetinib, cobimetinib, selumetinib, or trametinib. In further embodiments, the HER2 inhibitor is neratinib, trastuzumab, dacomitinib, lapatinib, tucatinib, or pertuzumab. In further embodiments, the CDK4/6 inhibitor is Ribociclib. In further embodiments, the HDAC inhibitor is CAY10603, AR-42, In further embodiments, the BCL2 inhibitor is Venetoclax, in further embodiments, the PARP inhibitor is Niraparib. In further embodiments, the ERK inhibitor is ERK_6604. In yet further embodiments, the JAK inhibitor is AZ960.

In some embodiments, an effective amount of the agent is administered to the patient.

In some embodiments, the agent is administered to the patient at a dose of about 0.1 mg to about 5000 mg. In further embodiments, the agent is administered to the patient at a dose of about 1 mg to about 1000 mg. In further embodiments, the agent is administered to the patient at a dose of about 1 mg to about 500 mg. In further embodiments, the agent is administered to the patient at a dose of about 1 mg to about 100 mg. In some embodiments, the agent is adminstered to the patient at a dose of about 25 mg, about 50 mg, about 100 mg, about 150 mg, about 200 mg about 250 mg, or about 500 mg.

In some embodiments, the agent is administered once, twice, three, four, five, or six times a day.

### Methods of Treatment

Provided is a method of treating a cancer patient, the method comprising administering an effective amount of an agent to the patient, wherein the patient has a cancer cell or tumor having an iGenSig score for the agent that indicates that the cancer cell or tumor will respond to the agent.

In some embodiments, the iGenSig score is calculated as described elsewhere herein. The iGenSig method generates prediction scores based on the set of redundant genomic features from labeled genomic datasets of therapeutic responses, and then reduce the effect of feature redundancy via adaptively penalizing the redundant features detected in specific samples based on their co-occurrence assessed using unlabeled genomic datasets for large cohorts of human cancers.

In some embodiments, the agent comprises a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil, or injectable organic esters. In some embodiments, when such pharmaceutical compositions are for human administration, particularly for injection or implantation, the aqueous solution is pyrogen-free or substantially pyrogen-free.

Excipients can be chosen, for example, to effect delayed release of an agent or to selectively target one or more cells, tissues or organs.

The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration.

A pharmaceutically acceptable carrier can contain a physiologically acceptable compound or mixture that acts, for example, to stabilize, increase solubility or to increase the absorption of an agent that is to be administered to a patient. The choice of a pharmaceutically acceptable carrier, including a physiologically acceptable compound or mixture, depends, for example, on the route of administration.

In some embodiments, the agent can be administered to the subject by any number of routes of administration including, for example, orally, absorption through the oral mucosa (e.g., sublingually), anally, rectally or vaginally (e.g., as a pessary, cream or foam), parenterally (including intramuscularly, intravenously, subcutaneously or intrathecally as, for example, a sterile solution or suspension), nasally, intraperitoneally, subcutaneously, transdermally (e.g. a patch), and topically (e.g., cream, ointment, spray, eye drop). The agent may also be formulated for inhalation.

In some embodiments, the agent is an EGFR inhibitor, a HER2 inhibitor, a CDK 4/6 inhibitor, a HDAC inhibitor, a BCL2 inhibitor, a JAK inhibitor, a PARP inhibitor, a ERK inhibitor, a MEK inhibitor, a BRAF inhibitor, irinotecan, topotecan, paclitaxel, 5-FU, Vincristine, Venetoclax, Epirubicin, or combinations thereof.

In further embodiments, the EGFR inhibitor is erlotinib, lapatinib, Afatinib, AZD3759. In further embodiments, the BRAF inhibitor is encorafenib, vemurafenib, or dabrafenib. In further embodiments, the MEK inhibitor is binimetinib, cobimetinib, selumetinib, or trametinib. In further embodiments, the HER2 inhibitor is neratinib, trastuzumab, dacomitinib, lapatinib, tucatinib, or pertuzumab. In further embodiments, the CDK4/6 inhibitor is Ribociclib. In further embodiments, the HDAC inhibitor is CAY10603, AR-42, In further embodiments, the BCL2 inhibitor is Venetoclax, in further embodiments, the PARP inhibitor is Niraparib. In further embodiments, the ERK inhibitor is ERK_6604. In yet further embodiments, the JAK inhibitor is AZ960.

In some embodiments, an effective amount of the agent is administered to the patient.

In some embodiments, the agent is administered to the patient at a dose of about 0.1 mg to about 5000 mg. In further embodiments, the agent is administered to the patient at a dose of about 1 mg to about 1000 mg. In further embodiments, the agent is administered to the patient at a dose of about 1 mg to about 500 mg. In further embodiments, the agent is administered to the patient at a dose of about 1 mg to about 100 mg. In some embodiments, the agent is adminstered to the patient at a dose of about 25 mg, about 50 mg, about 100 mg, about 150 mg, about 200 mg about 250 mg, or about 500 mg.

In some embodiments, the agent is administered once, twice, three, four, five, or six times a day.

### EXAMPLES

### Materials and Methods

### Data Retrieval

The drug response data, gene expression data, and mutation data are from the Genomics of Drug Sensitivity in Cancer Project (GDSC), and the Cancer Cell Line Encyclopedia (CCLE) as of September 2018. The GDSC and CCLE gene expression data are retrieved from ArrayExpress (E-MTAB-783) and NCBI GEO (GSE36133) respectively, and normalized using Robust Multi-Array Averaging (RMA)³. Drug sensitivity data, mutation data, and cell line annotations were downloaded from the GDSC (www.cancerrxgene.org/downloads) or CCLE (www.broadinstitute.org/ccle) websites. The newly released batch 2 drug sensitivity dataset are downloaded from GDSC website as of May 2021. The TCGA Pan-cancer gene expression and mutation datasets were retrieved from UCSC Xena browser (xenabrowser.net). The gene expression data for clinical trials are obtained from Gene Expression Omnibus (GEO). These include BATTLE trial, (GSE33072), Swiss SAKK 19/05 trial (GSE37138), multi-center clinical study carried out by the French CIT program (GSE39582), multi-center taxane treated stage 1-111 basal-like breast cancer patient cohort (GSE25055 and GSE25065). The gene expression and mutation data for the PDX tumors were retrieved from the supplementary dataset of the original publication⁴.

### Extracting Genome-Wide Expression and Mutation Features for Cell Line and Tumors

Based on gene expression and somatic point mutation datasets, genome-wide differential gene expression (DGE) and mutation features were extracted and an integrated genomic feature file was generated. For gene expression datasets, quantile normalizations were performed and genes with standard deviations of less than 20% percentile are filtered. We then calculated log 2 transformed fold changes of the expression values compared to the trimmed mean of expression values (excluding the 10% largest and 10% smallest values). To eliminate zero values during log 2 transformation, we added 1 to the expression value across all cell lines or tumors. Based on the mean and standard deviation (SD) of fold changes, we assigned the cell lines or tumors into the following overlapping groups: ‘Up_Level1’ group with the fold change above Mean+1*SD for a given gene; ‘Up_Level2’ group with the fold change above Mean+2*SD; . . . , and ‘Up_Level6’ group with the fold change above Mean+6*SD Likewise, ‘Down_Level1’, ‘Down_Level2’, . . . and ‘Down_Level6’ grouped cell lines based on Mean−1*SD, Mean−2*SD, and Mean−6*SD. The 12 ‘Levels’ were labeled as genotypic features for each given gene and the binary genomic features are compiled as a Genomic Matrix Transposed (GMT) file format. Similarly, we extracted binary genomic features to represent point mutations. The mutation hotspots and nonsynonymous somatic mutations such as missense, nonsense, and frame shift are assigned as mutation features. Each recurrent mutation hotspot and each recurrently mutated gene were assigned as separate features.

### Defining Drug Responses of Cancer Cell Lines

Drug responses of cancer cell lines are represented by the area under dose-response curve (AUC) in GDSC or the area over the dose-response curve (Act Area) in CCLE^{5, 6}. The skewness of the AUC measurements for each drug in the GDSC dataset was first tested. A negative skewness distribution indicates that the drug has high AUC measurements (lack of responses) in most of the cell lines, but low AUC measurements (sensitive responses) in a small subset of the cell lines, and a lower level of skewness indicates higher level of outstanding responses. To ensure the drugs have sufficient outstanding responders for training and testing the algorithm, the GDSC drugs with negative skewness and more than 20 sensitive cell line subjects are included in the iGenSig modeling described herewith. Sensitive drug responses of cell lines were defined based on Act Areas using the water fall method described in the CCLE study⁵. The Act Area measurements for CCLE or GDSC cell lines for a given compound are sorted in ascending order to generate a waterfall distribution. The cut-off for defining sensitive subjects was determined as the maximal distance to a line drawn between the start and endpoints of the distribution. The cut-off for non-responders was determined as ‘median of Act Area—median absolute deviation (MAD).’ The cell lines with Act Area above the sensitivity cut-off were labeled as drug-sensitive and below the resistance cut-off were labeled as drug-resistant. The cell lines with Act Areas between the cut-offs for drug sensitivity and resistance were labeled as intermediate.

### Calculating Feature Weights and Selecting Significant Genomic Features

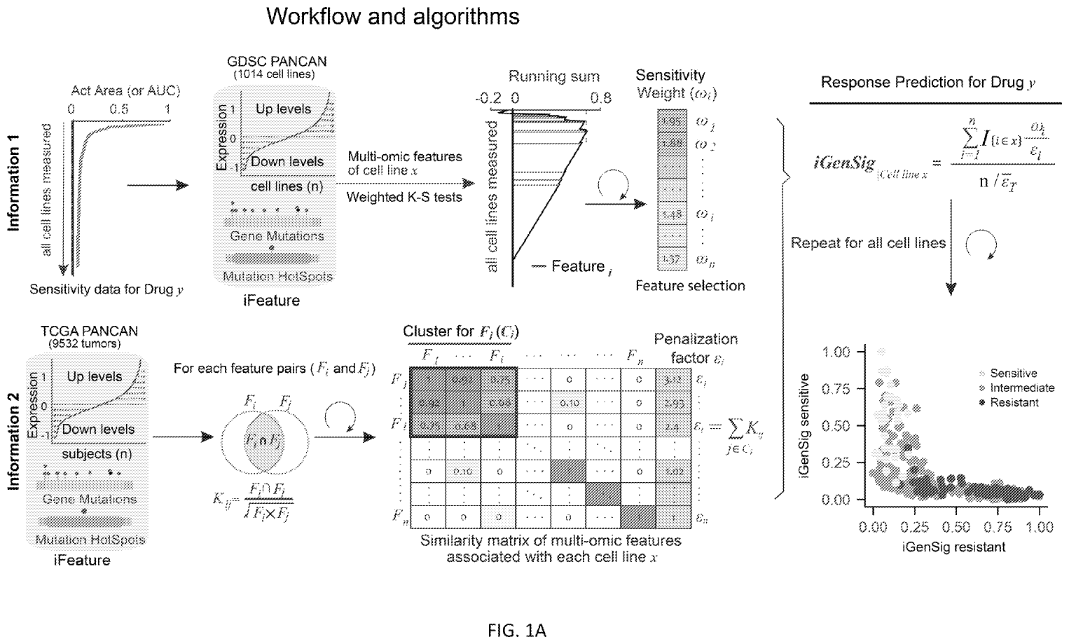

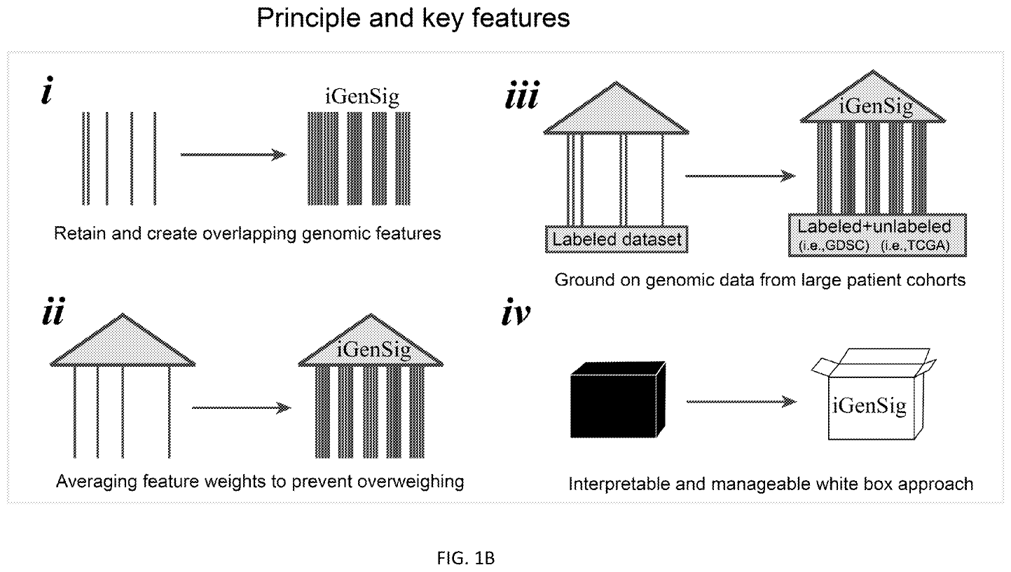

To define the weight (ωₗ) of each genomic feature in predicting sensitive drug responses, we leveraged the weighted Kolmogorov-Smirnov (WKS) statistics⁷ to test the enrichment of the feature-positive cell line in the cell line panel sorted in descending order based on Act Area (FIG. 1A). The enrichment score (ES) for each genomic feature is calculated as previously described⁷. To prevent bias, the genomic features defining fewer than 5 cell lines were excluded during the calculation of GenSig scores. Likewise, we calculated the weights for each genomic feature in predicting resistant drug responses based on the cell line panel sorted by AUC in descending order. To prevent overfitting, for a give cell line x in the training set, cell line x is excluded from calculating the ES scores for the genomic features associated with cell line x. We assessed the significance of the observed ES by comparing that to the random ES scores calculated by random features with the same numbers of positive cell lines. This step was repeated until 2000 random enrichment scores were calculated, then the normalized enrichment score (NES) was calculated by:

NES=ES/mean(ES_{random}) 1)

The p-values were determined based on the chance of random ES scores to be above the observed ES score for feature i, and the false discovery rate (FDR) were calculated using R package “qvalue”. In this study, we used a universal FDR q-value cutoff of 0.1 to select significant genomic features for calculating iGenSig scores. This parameter, however, can be tuned for different drugs as the signal to noise levels of these predictive genomic features could be different for different drugs. Furthermore, we observed that some of the genes have both upregulation and downregulation features ranked as significant for predicting the sensitive drug response. We thus filtered the genes that have both upregulated features with FDR <0.1, and downregulated features with FDR <0.3, and the genes that have both downregulated features with FDR <0.1, and upregulated features with FDR <0.3. On the other hand, some of the genes have only level-1 DGE features selected as significant based on FDR <0.1, but none of their corresponding high levels of DGE features have an FDR more than 0.3 even if they define more than ten cell lines. These genomic features represent noises and are thus filtered as well.

The Algorithms for Penalizing Feature Redundancy and Methods for iGenSig Modeling

To prevent the inflation of iGenSig scores from feature redundancy, we leveraged the TCGA Pan-Cancer RNA-seq and exome datasets to assess the co-occurrence between genomic features associated with each cell line and generated the cosine similarity matrix of genomic features based on Otsuka-Ochiai coefficient between these features (Kᵢⱼ). We then performed clustering of the cosine similarity matrix based on Ward's method (D2) using the R module “hClust”. The correlated feature groups are then determined based on an adaptive dynamic cluster detection method², using the parameters: dynamic.method=“hybrid”, cutTree.depth=2, and minClusterSize=40. We then introduced a penalization factor (ε) which is calculated for each genomic feature i based on the similarity indices of the colinear genomic features associated with a given cell line ₓ and of the same cluster as feature i:

εᵢ=Σ_{j∈Cluster}_{ᵢ}Kᵢⱼ 2)

Where Kᵢⱼ is the Otsuka-Ochiai coefficient between feature i and a given genomic feature j from the same cluster group as feature i associated with cell lineₓ. To eliminate the cumulative effect of small overlaps between genomic features, the Otsuka-Ochiai coefficients were adjusted to 0 if Kᵢⱼ<0.1. Here e, is an estimator of redundancy among the genomic features of the same cluster group associated with cell line ₓ. The penalization factor ranges from 1 (all genotypes are completely different each other) to n (all genotypes are the same). We then penalized the weight ωᵢ using εᵢ, resulting in Effective Weight (EW):

$${EW}_{i} = \frac{{ω}_{i}}{{ε}_{i}} \qquad 3 )$$

The trimmed mean of εᵢ (trim=0.3) was then used to calculate the Effective Feature Number (EFN):

$${EFN}_{i} = \frac{n}{{\overline{ε}}_{T}} \qquad 4 )$$

Finally, the GenSig score of the given cell line, is computed as:

$${GenSig}_{\vert Cell {Line}_{x}} = \frac{{\sum }_{i = 1}^{n} {EW}_{i}}{{EFN}_{i}} = \frac{{\sum }_{i = 1}^{n} {I}_{\{ i ∈ x \}} \frac{{ω}_{i}}{{ε}_{i}}}{\frac{n}{{{ε}^{{\_}^{}}}_{T}}} \qquad 5 )$$

The sensitive and resistant iGenSig scores are calculated separately based on the significant genomic features selected for predicting sensitive or resistant responses. The sensitive iGenSig scores are used for assessing the performance of the iGenSig models on predicting sensitive cell lines and patient subjects. Thus, the iGenSig scores labelled in the figures refer to the sensitive iGenSig scores unless otherwise noted.

Benchmarking the Performance of the iGenSig Algorithm

To benchmark the performance of the iGenSig algorithm in determining drug sensitivity, 20% of GDSC cell lines treated by a specific drug were selected as internal test set. The rest of 80% cell lines were assigned as train set and performed this randomized sampling for 5 times. The distributions of drug sensitive and resistant cell lines were required to be balanced between the train and test set in each sampling. The CCLE dataset was used as external validation set of our predictive models to assess their applicability to an independent dataset. The area under ROC curve (AUROC) of the iGenSig scores was calculated based on the binary response of the cell lines determined based on the sensitive cutoff discussed above, and the optimal cut points of iGenSig scores are determined using the R module “coords” of the “pROC” package. The cell line subjects were divided into sensitive cell lines and other cell lines that include both intermediate and resistant cell lines, and the sensitive iGenSig scores are used when assessing the predictive values of the iGenSig models.

To test if the iGenSig predictions rely on the genomic features of the primary drug targets, we removed the drug target gene features for Erlotinib, Lapatinib, or Sorafenib from GDSC and CCLE genomic feature sets. The iGenSig models were then built based on the genomic features devoid of drug targets and assessed their performance on internal test set (20% of GDSC cell lines) or external validation set (100% of CCLE cell lines). To examine if excluding the hematologic cancer cell lines from the GDSC training dataset can improve the prediction performances of iGenSig models on the drug sensitivity of CCLE solid cancer cell lines, the leukemia, lymphoma, and myeloma cell lines were removed from the GDSC dataset when performing the modeling for the shared 14 drugs, and then the models were applied to CCLE solid cancer cell lines.

### Meta-Analyses of Clinical Trial and Prospective Clinical Study Datasets

To examine the predictive values of the iGenSig models on patient subjects, we compiled five clinical trial or prospective clinical study datasets. The iGenSig model for the specific drug tested on a given treatment arm of the trial are developed based on GDSC dataset, and then applied to the genomic features of the clinical trial datasets. These include the models for Erlotinib (Drug ID: 1), Sorafenib (Drug ID: 30), 5-Fluorouracil (Drug ID: 179), and Paclitaxel (Drug ID: 1080). The uses of clinical endpoints are dependent on the clinical information provided by the authors of the original publications. Overall survival (OS) is the preferred endpoint of choice, followed by pathologic complete response (pCR). Other endpoints will be used in the analysis if OS and pCR are not available. For survival analysis, a data-driven cut point of high iGenSig scores was determined as previously described¹, using the R-package “maxstat”, and the P-value are calculated based on log-rank tests.

### Deep Learning and Machine Learning Methods

Deep learning method autoencoder⁹ was applied to perform unsupervised representation learning for dimensionality reduction and machine learning prediction algorithms for supervised learning of therapeutic responses using the low dimensional features generated by autoencoder and compared their prediction performances with the iGenSig method. The Autoencoder model was developed using the same genome-wide gene expression and mutation features applicant compiled, and we the same training, internal testing, and external validation sets of cell line models as in iGenSig modeling were used. The models are developed based on the 80% of GDSC datasets (5 permutated training sets). The autoencoder model was built with three hidden layers with the unit sizes in each layer designed based on a previous report¹⁰. The unsupervised representation of the genomic correlates were applied to supervised learning methods including elastic net, artificial neural network, Random Forest (RF), and support vector machine (SVM) for prediction modeling. Elastic net is a regression method that combines lasso and ridge regularization with the two hyperparameters, alpha and lambda. Alpha is a mixing parameter to define the relative weight of the lasso and ridge penalization terms and lambda determines the amount of shrinkage¹¹. Alpha with the best tuning was identified and was optimized for predictive performance over a range of lambdas. Regression was performed using the glmnet R package (ver. 4.0.2). RF regression model was implemented using randomForest R package (ver.4.6.14). It was specified for 1,000 trees to grow and it was ensured every object got predicted multiple times. SVM with linear kernel method, ‘svmLinear2’, was used, provided by caret R package (ver. 6.0.86). tuneLength=10 was specified in the tuning parameter grid and accuracy metric. For ridge regression, the glmnet R package was used for binomial classification based on the original high-dimensional binary genomic features. For tuning the model, the glmnet function was allowed to compute its own array of lambda values and the optimal ridge model is determined based on “lambda.1se”. For all modeling methods, one model is developed for each drug based on each permutation set, which are then applied to the clinical trial datasets. To match the genomic features, the genomic features are set to zero if they do not present in the validation sets as in the iGenSig modeling.

### Pathway Enrichment Analysis for Integral Genomic Signature

To identify the pathways characteristic of the integral genomic signature for Erlotinib resistance modeled from the GDSC dataset, the genes involved in the iGenSig signature were first extracted and then classified into positive contributing genes and negative contributing genes. The positive contributing genes are defined as upregulated genes or genes with hotspot mutations. The negative contributing genes are defined as downregulated genes or mutated genes without mutation hotspots. The pathways enriched in the positive or negative contributing genes for predicting Erlotinib or 5-FU sensitive responses are analyzed by the Concept Signature Enrichment Analysis (CSEA) developed in our previous study¹². The resulting top pathways are disambiguated via correcting the crosstalk effects between pathways, to reveal independent pathway modules¹³. A p-value <0.01 is used as cutoff for disambiguation. The functional associations between the significant pathways are then assessed using our CSEA method as we previously described¹², and the CSEA scores are then scaled between −1 and 1 and visualized using correlogram. The pathway network was visualized using the ‘igraph’ R package (ver. 1.2.4.2)

### Code Availability

The R modules for iGenSig modeling are available through github: github.com/wangxlab/iGenSig/.

### Example 1: Development of the Integral Genomic Signature Approach Based on Genomic Dataset of Drum Sensitivity

A new class of methods for big data-based precision medicine called integral genomic signature (iGenSig) analysis is proposed herein, which is designed to provide more robust clinical decision support with higher transparency, outstanding resilience, and cross-dataset applicability (FIGS. 1A-1B). Due to the high dimensionality of genomic features, a common practice for big data-based modeling is to reduce the dimensionality of genomic features via removing redundant variables highly correlative with each other as for gene expression signature panels, or creating synthetic features as for machine learning approaches¹⁴. It is proposed herewith that the redundancies within high-dimensional features can in fact overcome sequencing errors and bias especially when there is a loss of detection of a subset of correlates. Applicant defines the genomic features significantly predicting a clinical phenotype (such as therapeutic response) as genomic correlates, and an integral genomic signature as the integral set of redundant high-dimensional genomic correlates for a given clinical phenotype such as therapeutic response. The iGenSig analysis generates prediction scores based on the set of redundant genomic features from labeled genomic datasets of therapeutic responses, and then reduce the effect of feature redundancy via adaptively penalizing the redundant features detected in specific samples based on their co-occurrence assessed using unlabeled genomic datasets for large cohorts of human cancers from The Cancer Genome Atlas (TCGA) (FIG. 1A). This allows for preserving redundant genomic features as well as introducing de novo redundant genomic features during the modeling, while preventing the feature redundancy from flattening the scoring system. With this method, it is speculated that if a subset of the genomic features was lost due to sequencing biases or experimental variations, the redundant genomic features will help sustain the prediction score. More important, it is expected that the second genomic information obtained from unlabeled large cancer cohorts will substantially improve cross dataset applicability of the iGenSig models, particularly on clinical trial datasets. On the other hand, iGenSig modeling utilizes the average correlation intensities of significant genomic features detected in specific samples to diminish the effect of false positive detection resulting from sequencing errors and overweighing. This method also prevents overfitting through dynamically adjusting the feature weights for training subjects. This approach will be more interpretable and controllable than machine learning or deep learning approaches and will prevent known issues for AI based prediction modeling based on multi-omics big data. Thus, iGenSig is a simple, white box solution with an integral design to tolerate sequencing errors and bias for big data-based precision medicine. The principle and key features of iGenSig modeling are summarized in FIG. 1B.

To develop the iGenSig modeling, the drug sensitivity measurements of chemical perturbations, gene expression profiling data, and exome sequencing data for 989 cancer cell lines released by Genomic Datasets of Drug Sensitivity (GDSC) were utilized. For the drug response measurements, high Act Area, the area above the fitted dose response curve (or 1-AUC), were utilized to define a sensitive drug response, and high AUC, the area under the dose curve, were utilized to define a resistant response. According to literature, the AUC and Act Area are much better quantifiers of drug responses than IC50¹⁵. To uniform multi-OMIC features, a Genomic-feature Matrix Transposed (GMT) format was formulated for compiling binary multi-OMIC features, similar to that used for compiling gene concepts^{12, 16}. Using this format, the expression profiling data and exome sequencing data from GDSC were analyzed and an integrated dataset was compiled combining the genomic features including upregulated genes, downregulated genes, mutated genes, and mutation hotspots. To increase the cross-dataset applicability of the iGenSig models, de novo feature redundancy was intentionally introduced by generating overlapping levels of differentially expressed gene lists (FIG. 1A). Significant genomic correlates were selected using a weighted Kolmogorov-Smirnov (K-S) test that ranks the enrichment of each genomic feature in the cell line panel sorted decreasingly by Act Area or AUC, similar to that implemented by Gene Set Enrichment Analysis (GSEA)⁷. The TCGA Pan-Cancer RNAseq and exome dataset for 9532 tumors was leveraged to quantify the cooccurrence between genomic features associated with each cell line based on similarity measures, which were then used to calculate a redundancy penalty score for each genomic feature.

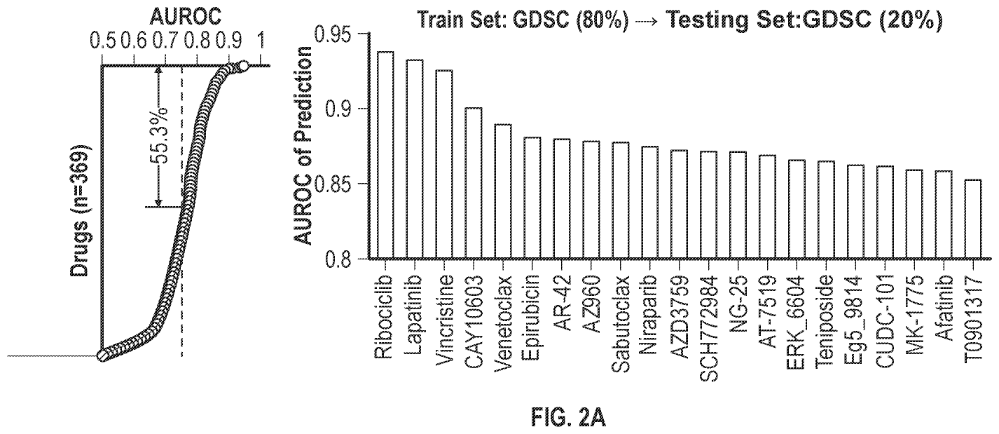

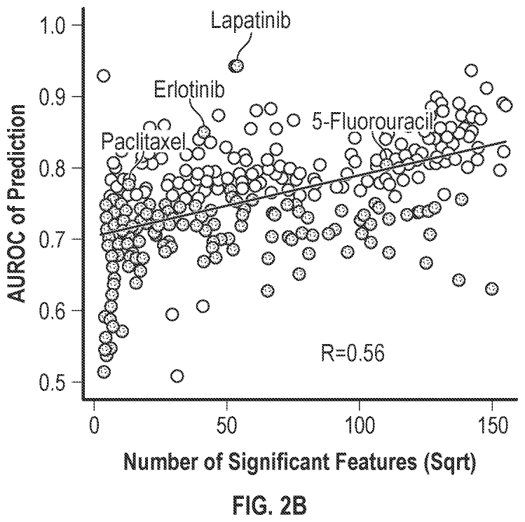

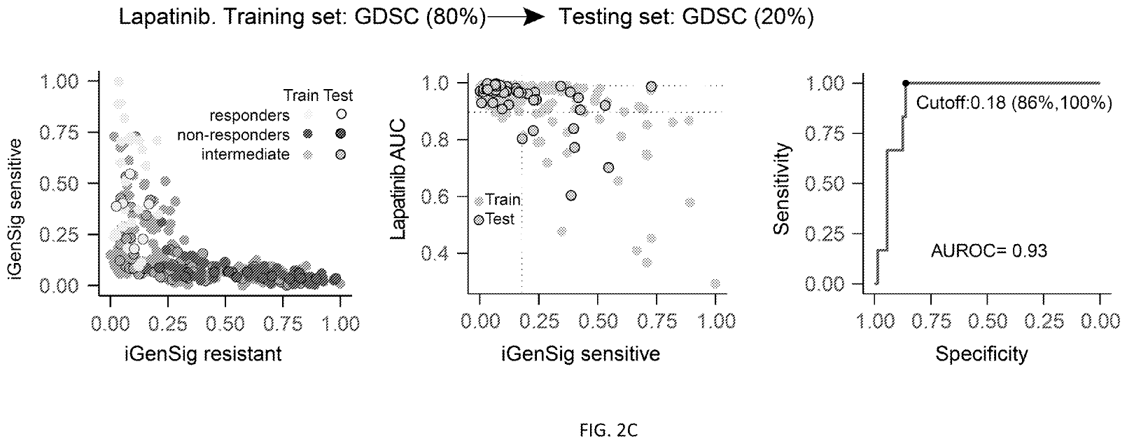

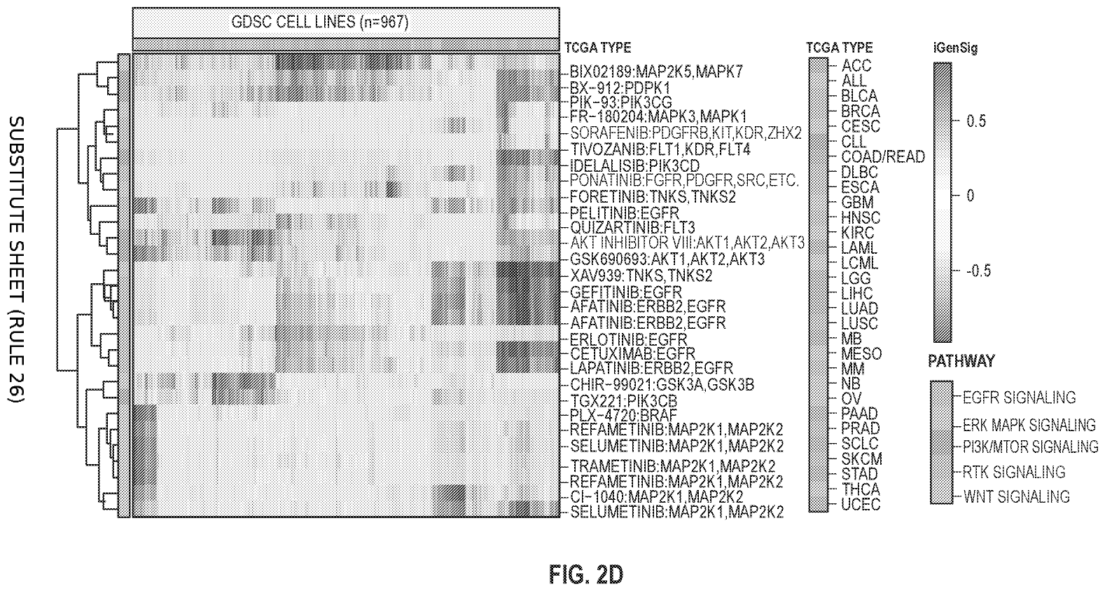

To prevent the bias from overfitting, a random collection of 80% GDSC cell lines were used as train set and the rest 20% were used as internal test set for assessing the performance of the model. A total of five train/test sets are generated for modeling through random permutations. iGenSig modeling was performed for 369 drugs that elicit a negatively skewed drug response distribution in cancer cell lines indicating narrow effect of outstanding responses as observed for most targeted therapies, and have at least 20 sensitive cell line subjects indicating the availability of outstanding responders. To benchmark the performance of the models, the cell lines were discretized into drug sensitive and non-sensitive groups based on a water fall method established in a previous study⁵, and the Area Under ROC Curve (AUROC) was calculated for each drug. As a result, 204 drugs showed an AUROC >0.75 on the testing sets (55.3%), and 21 drugs showed an AUROC>0.85 (FIG. 2A). Many of the top performing drugs are FDA approved chemotherapy or targeted therapy agents for cancer treatment, such as Ribociclib, Lapatinib, Vincristine, Venetoclax, Epirubicin, Niraparib, and Afatinib. The top performing drug models include targeted therapies against well-known cancer targets such as CDKs, ERBBs, HDAC, BCL2, JAKs, PARP, ERK, etc, and Ribociclib, Lapatinib, and Vincristine presented the best performing models with an average AUROC of 0.93-0.94. The predictive powers of the iGenSig models appear to obviously correlate with the number of available genomic correlates for each drug (Spearman R=0.56, FIG. 2B), suggesting that the iGenSig models rely on the available genomic information that can predict the drug responses. The iGenSig scores negatively correlate with the AUC drug measurements in cell lines with a similar trend in both training and testing sets as exemplified by the Lapatinib model (FIG. 2C), suggesting that iGenSig modeling do not overfit toward training set as opposed to AI-based methods. Next, the drugs that target kinase signaling were clustered based their iGenSig scores in GDSC cell lines, which resulted in distinctive clustering of the drugs targeting the same or similar kinases (FIG. 2D). Interestingly, outstanding response predictions for BRAF/MEK inhibitors are preferentially enriched in melanoma cell lines, while other drugs such as EGFR inhibitors exhibit cancer type agnostic iGenSig scores, consistent with the tumor-type related clinical activities of these drugs.

### Example 2: iGenSig Models Show No Performance Loss on Independent Validation Dataset for Drug Sensitivity

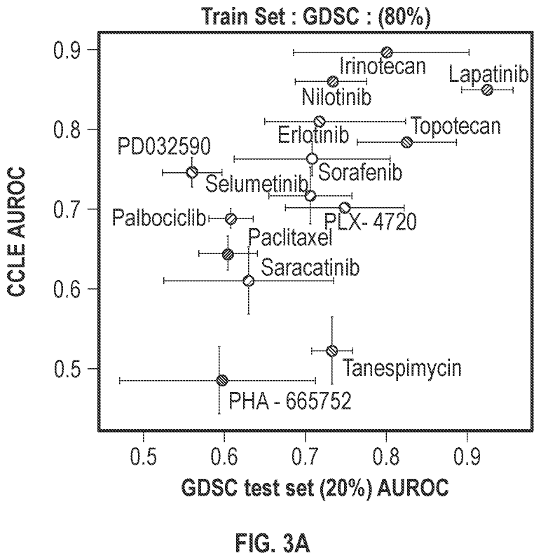

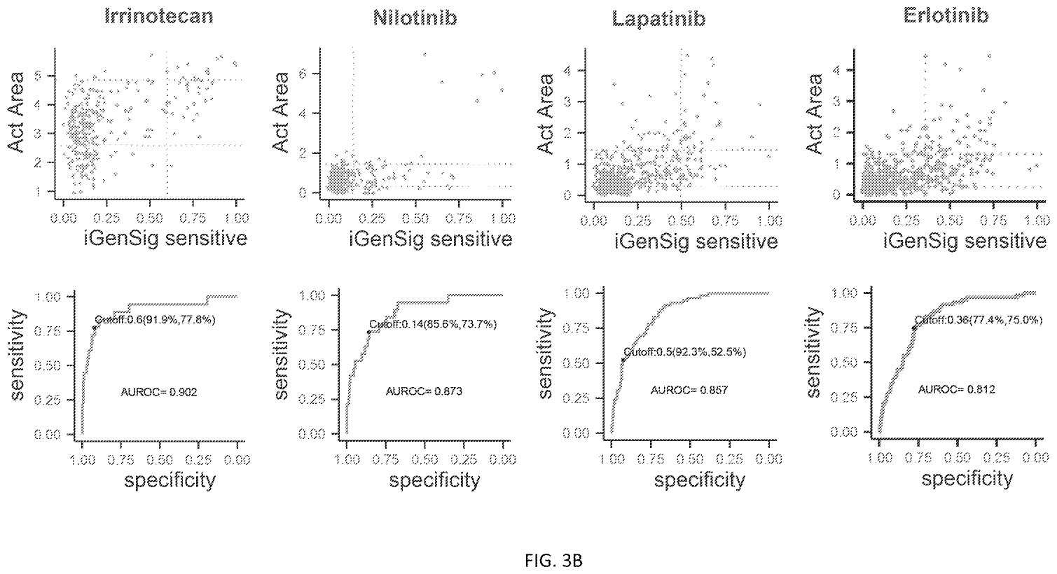

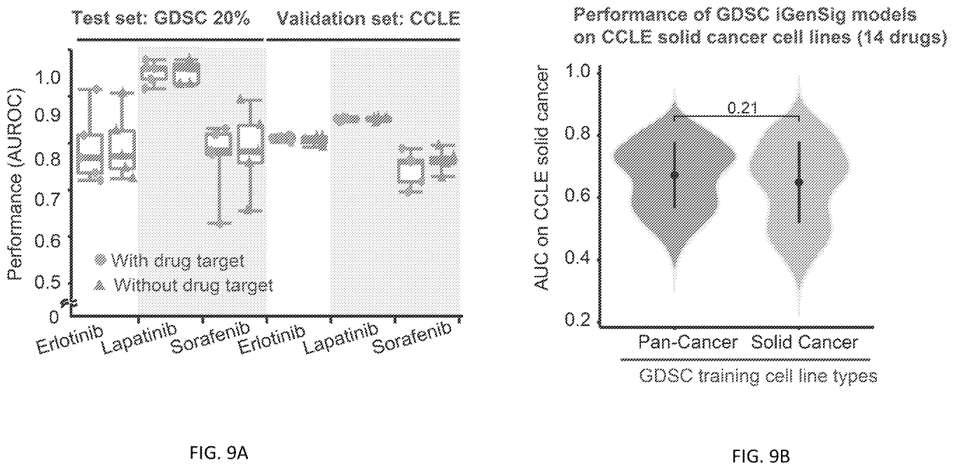

To assess the cross-dataset performance of iGenSig models, the RNAseq and exome sequencing data from the Cancer Cell Line Encyclopedia (CCLE) was analyzed. In total there are fourteen drugs measured by both CCLE and GDSC datasets. The results described herein showed that the predictive performance of iGenSig models on the CCLE dataset appear to correlate with their performance on the testing sets of the GDSC dataset (Pearson R=0.58, FIG. 3A). Using GDSC as training set and CCLE as validation set, the models for four drugs achieved AUROC of more than 0.8. These include Irinotecan, Nilotinib, Lapatinib, and Erlotinib, for which the AUROC for prediction are 0.902, 0.873, 0.857, and 0.812 respectively (FIG. 3B). Plotting the significant genomic features for Erlotinib in the two datasets revealed consistent integral genomic signature correlating with drug sensitive or resistant responses (FIG. 3C). This suggests that the modest consistency between the GDSC and CCLE measurements previously reported could be attributed to the number of cell lines screened by both GDSC and CCLE for which insufficient sensitive cell lines were screened in both projects, as suggested by the previously study¹⁷, or possibly due to the different cellular states under different cell culture conditions. It is interesting to note that the predictive performance of iGenSig models resulting from the permutated training sets on the CCLE validation dataset showed much lower deviations compared to that on the GDSC testing dataset (FIG. 3A). This may be attributed to the much smaller number of sensitive subjects in the GDSC testing datasets compared to the CCLE validation dataset. To test if the iGenSig predictions rely on the genomic features of the primary drug targets, the drug target genes for Erlotinib, Lapatinib, or Sorafenib were removed from GDSC and CCLE genomic feature sets. The iGenSig models were then built for these drugs based on the genomic features devoid of drug targets and their performance was assessed on GDSC internal test set or the CCLE validation set (FIG. 9A). The results showed that the performances of the iGenSig models were not affected by the absence of genomic features for known drug targets. Furthermore, it was examined if excluding the hematologic cancer cell lines such as leukemia and lymphoma from the GDSC training dataset can improve the prediction performances of iGenSig models on the drug sensitivity of CCLE solid cancer cell lines. Results, however, did not yield significantly improved performance of the fourteen drug models, but instead, this approach slightly decreased the overall performance (FIG. 9B). This suggests that there may be predictive genomic information gained from these hematologic cancer cell lines as well. The models developed from the Pan-cancer cell line dataset were thus used in the following analysis.

### Example 3: The iGenSig Model Predicts the Response of Patient Subjects to Erlotinib Treatment in the U.S. BATTLE Trial and Swiss SAKK 19/05 Trial

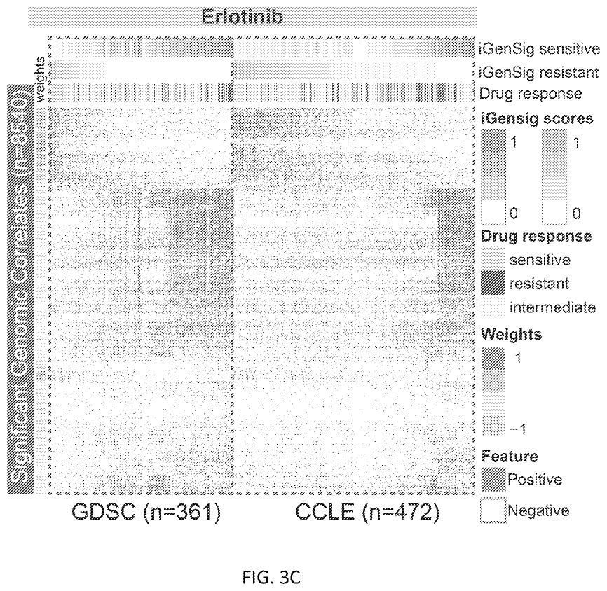

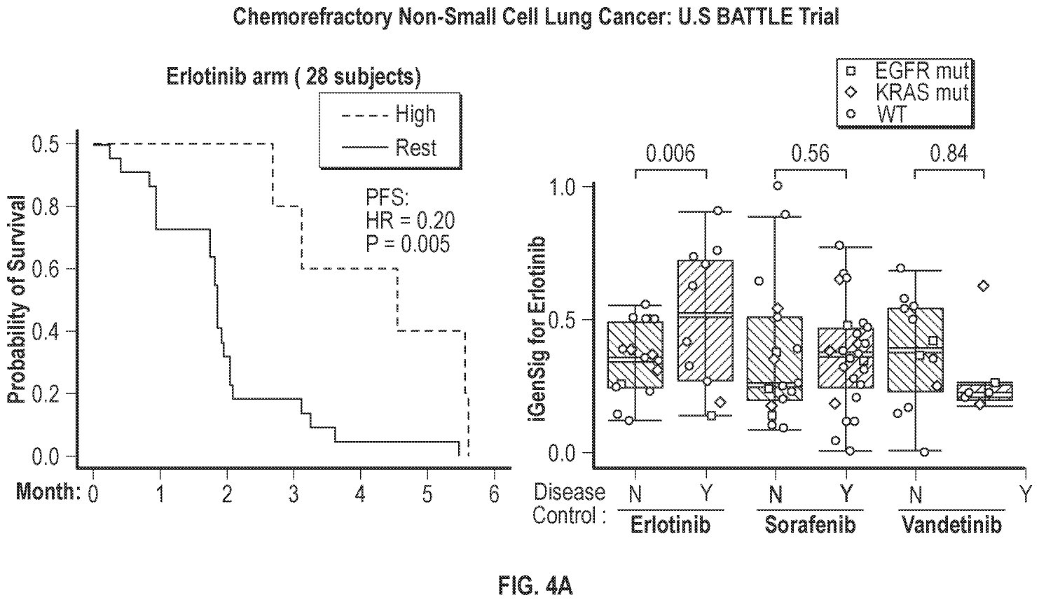

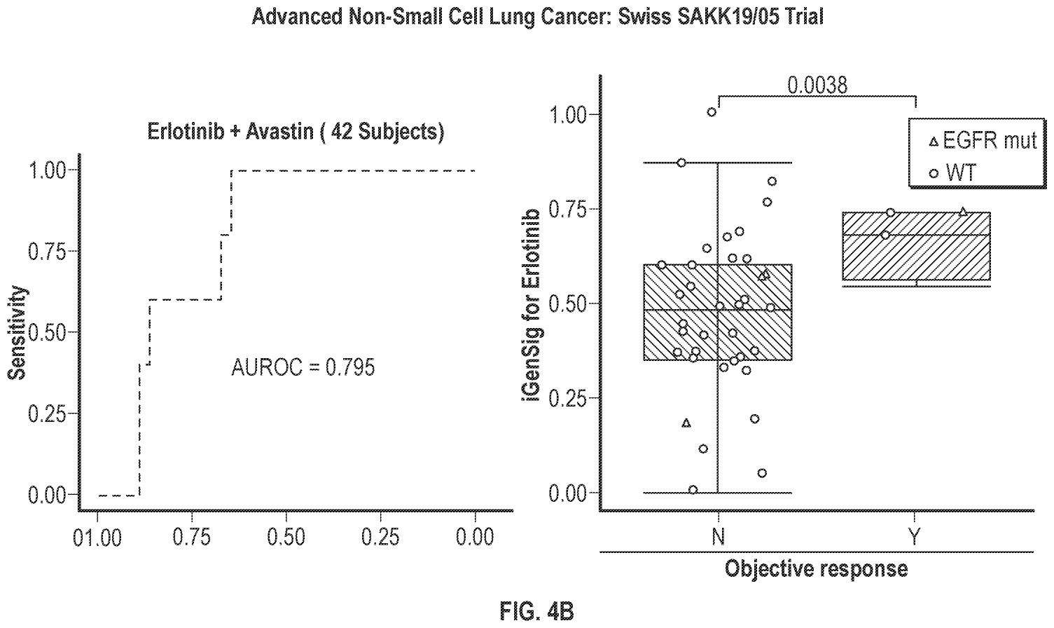

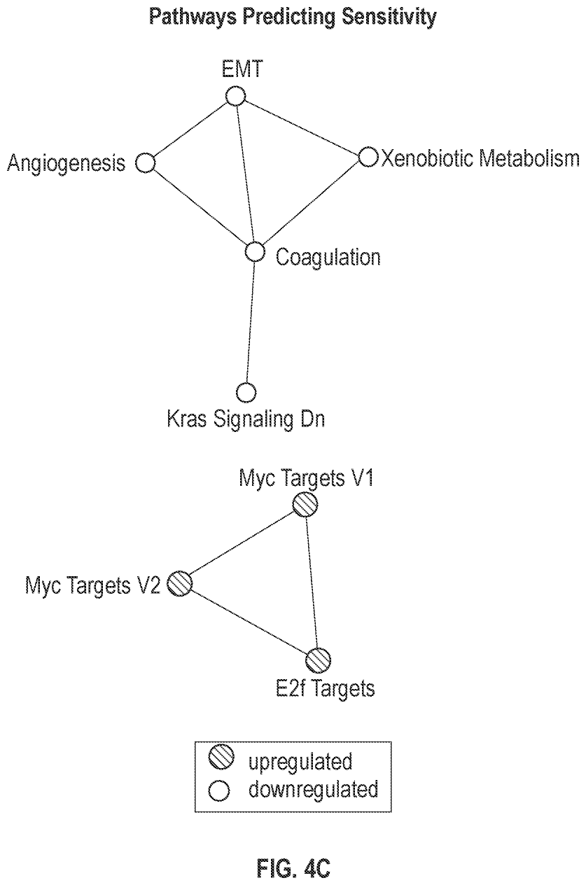

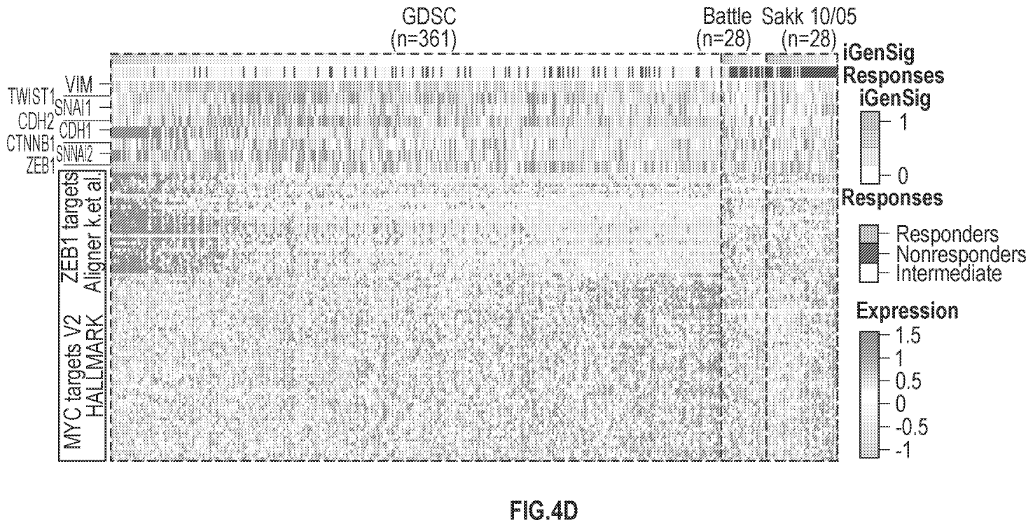

Next, the applicability of the GDSC iGenSig models on predicting therapeutic responses of patient subjects in clinical trials was tested. The availability of clinical trial datasets for EGFR inhibitors for which applicant's iGenSig models showed outstanding cross-dataset performance in the CCLE dataset was first examined. Most of the clinical trials for targeted drugs assessed their combinations with chemotherapies instead of monotherapies. Without wishing to be bound by theory, this may confound the outcome of drug response prediction. Literature investigation by the applicant revealed that a recent study of the BATTLE trial (GSE33072) profiled non-small cell lung cancer (NSCLC) tumors from 131 patients by gene expression array, among which 28 patients are treated with Erlotinib monotherapy, 47 patients are treated with Sorafenib monotherapy, and 20 patients are treated with vandetinib. Overall, the patient responses to Erlotinib in this trial are limited, and all patients treated with Erlotinib progressed within six months. Without wishing to be bound by theory, this may be due to the selection of pretreated chemorefractory NSCLC patients as enrollment criteria¹⁸. In spite of this, progression free survival analysis suggested that applicant's GDSC iGenSig model for Erlotinib significantly predicted the favorable response of these patients in the Erlotinib arm, with a hazard ratio of 0.2 (p=0.005, FIG. 4A, left). Among the three major treatment arms of this trial, the Erlotinib model showed specific predictive effect on the Erlotinib arm compared to the Sorafenib or Vandetinib arms (FIG. 4A, right).

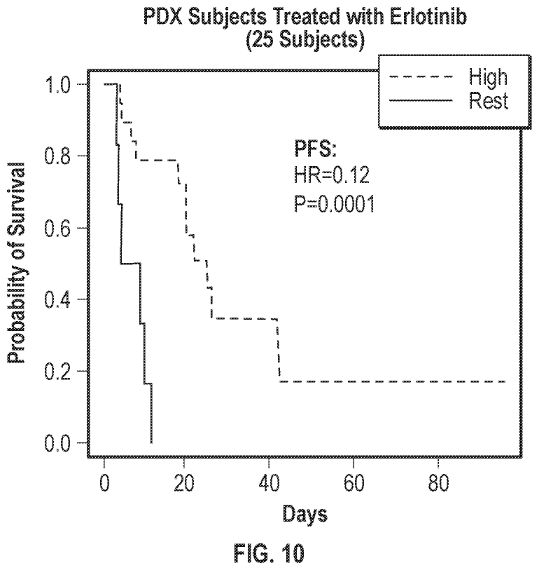

Next, the predictive value of this model was examined on a Swiss SAKK 19/05 trial that tested the combination of Erlotinib and bevacizumab (Avastin)¹⁹. Recent evidence suggested that addition of Bevacizumab to Erlotinib exhibits increased therapeutic efficacy. As bevacizumab alone is known to lack efficacy in lung cancer, this effect is thought to be the result of enhanced erlotinib activity¹⁹. The SAKK 19/05 trial is a multicenter single arm trial in previously untreated patients. The endpoint provided by this study is objective response, and no survival data are available. As a result, the GDSC iGenSig model for Erlotinib showed a predictive AUROC of 0.795 (FIG. 4B, left), and this predictive value is independent of EGFR mutation status (FIG. 4B, right). On the other hand, out of the four patients with EGFR mutated tumors, only the tumor showing highest iGenSig score exhibited objective response. This suggests that while EGFR inhibition is indicated for EGFR mutated patients, a subgroup of EGFR wild-type patients may derive significant benefit from EGFR inhibitors as well which could be identifiable by the iGenSig model. Moreover, in addition to clinical trial datasets, the iGenSig model was applied to a set of PDX models treated with Erlotinib and profiled with RNAseq and WXS, which revealed significant predictive value as well (HR=0.12, p=0.0001, FIG. 10). Taken together, these results support the utility of integral genomic signature modeling in predicting therapeutic responses of EGFR inhibition, and its outstanding cross-dataset performance.

### Example 4: Biological Interpretation of the iGenSig Model Yielded New Insights into the Predictive Signature Pathways with Clinical Relevance

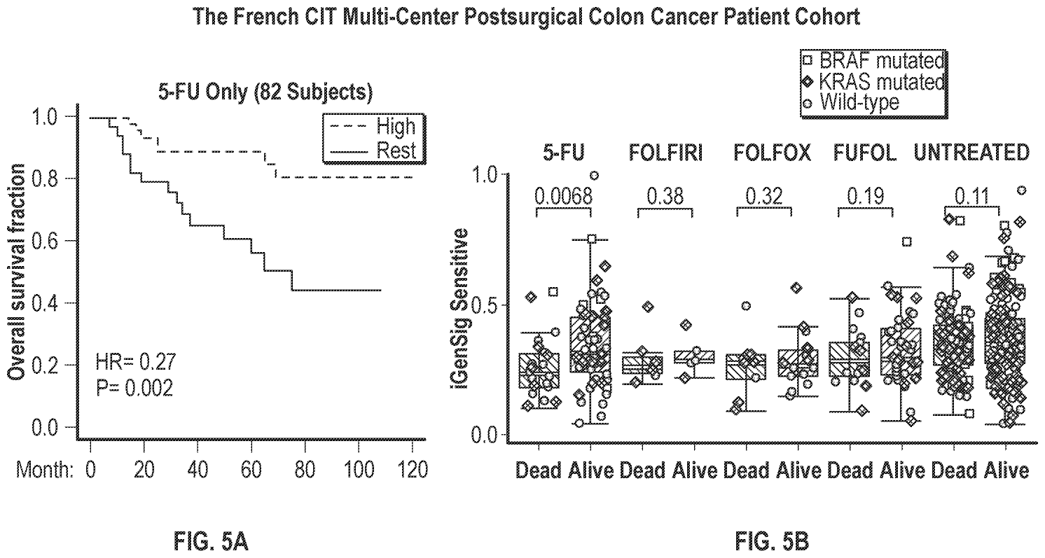

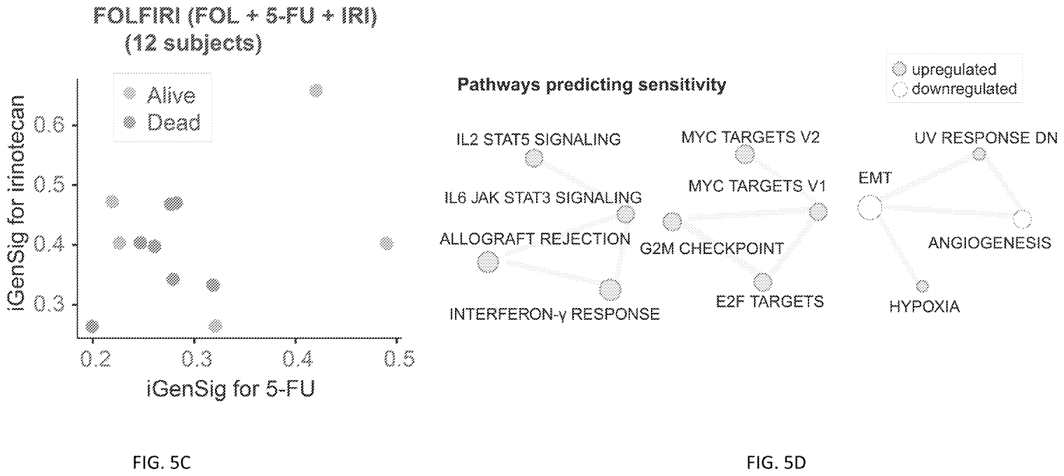

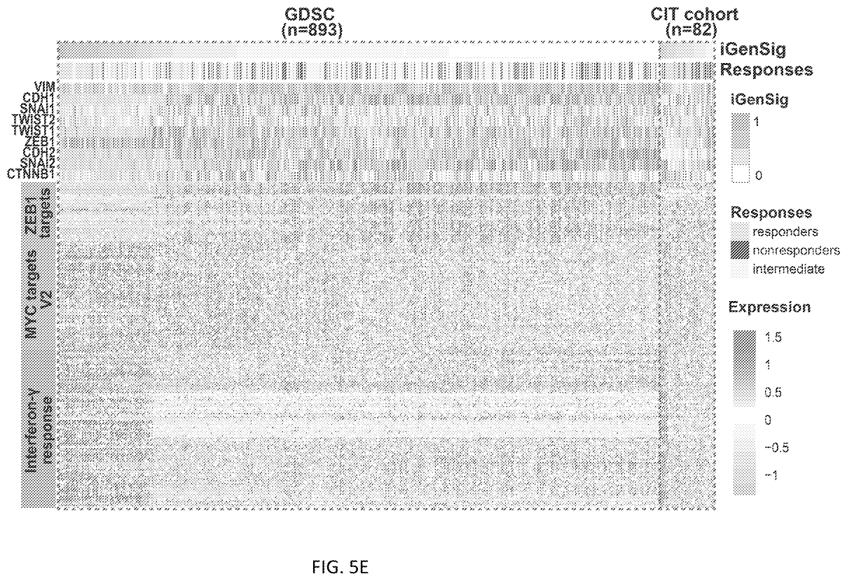

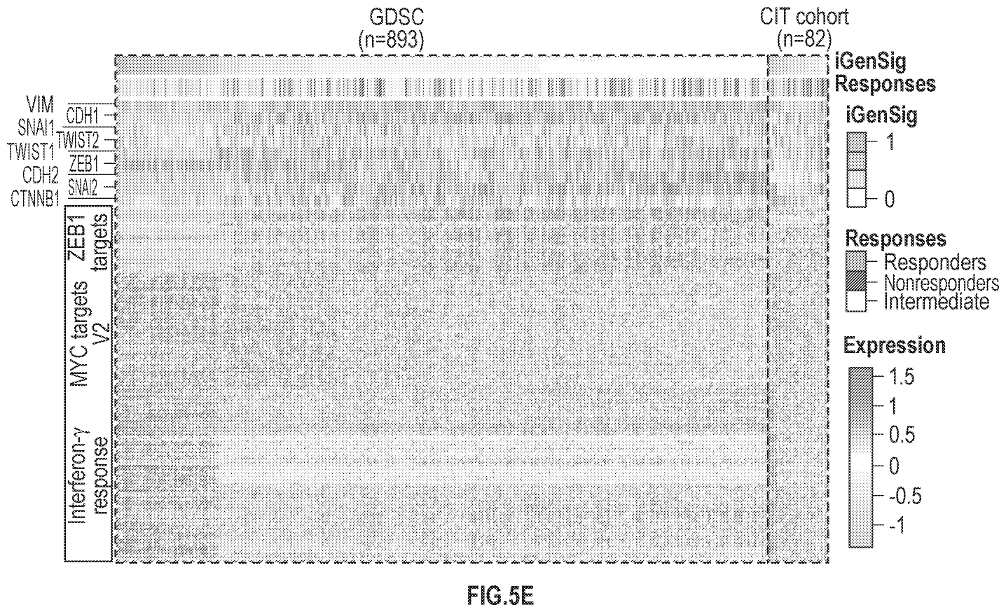

Since epithelial mesenchymal transition (EMT) has been previously reported to mediate EGFR resistance in the BATTLE trial study²¹, applicant wondered if the EMT signature contribute to the iGenSig predictions. The pathways characteristic of the integral genomic signature for Erlotinib sensitivity were examined in the iGenSig model. This can be achieved by extracting the genes contributing to the genomic features predicting sensitive response in the GDSC iGenSig model. The resulting gene list can be then used to explore the enriched pathways based on the concept signature enrichment analysis (CSEA) developed in applicant's previous study, which was designed for deep functional assessment of the pathways enriched in an experimental gene list¹². Results showed that the most significantly downregulated pathways characteristic of Erlotinib sensitivity signature include MYC and E2F target gene signatures (FIG. 5C). Consistent with this, amplification of MYC has been found to mediate resistance to EGFR inhibitors and targeting MYC has been proposed as a promising strategy to overcome acquired resistance²¹. On the other hand, the EMT pathway is ranked as one of the most significantly upregulated pathways in the Erlotinib resistance signature identified from GDSC cell lines, which contradicts the previous report²⁰.

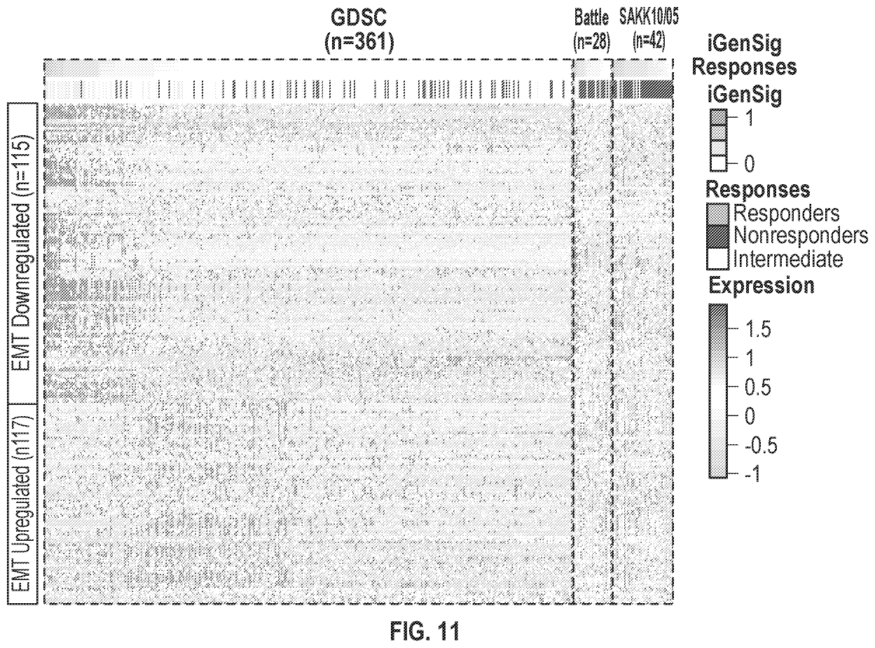

Without wishing to be bound by theory, this may be attributed to the content of the EMT signature that mixed both upregulated and downregulated genes in EMT. An upregulated EMT signature and a downregulated EMT signature were thus compiled based on a previous report²². Correlating these EMT signatures with the Erlotinib iGenSig scores revealed that the downregulated and upregulated EMT signatures are indeed enriched in the subjects with high or low iGenSig scores respectively in the BATTLE trial dataset (FIG. 11). However, in the GDSC Pan-Cancer cell line panel, both upregulated and downregulated EMT signatures are repressed in Erlotinib-resistant cell lines. This suggests that repression of both EMT signatures are characteristic of the Erlotinib resistant cell lines at Pan-cancer scale and explains the pathway results from CSEA analysis. Among the known EMT markers and transcription factors, overexpression of E-cadherin (CDH1) was observed in both sensitive cell lines and patient responders from BATTLE trial (FIG. 4D). Whereas overexpression of EMT markers such as N-cadherin (CDH2), Vimentin (VIM), and β-catenin (CTNNB1) are characteristic of the cell lines with intermediate sensitivity, and overexpression of ZEB1 is characteristic of Erlotinib resistant cell lines. In the BATTLE trial, overexpression of either β-catenin or ZEB1 are characteristic of subjects with low iGenSig scores. As ZEB1 is a transcriptional repressor, the correlation of ZEB1 target genes with the iGenSig scores was assessed. This revealed that downregulation of ZEB1 target genes is characteristic of both resistant cell lines and patient subjects in the two clinical trials (FIG. 4D). On the other hand, MYC target genes appear to be upregulated in the most Erlotinib resistant cell lines. Together, the results suggest that EMT is associated with reduced but intermediate response to Erlotinib whereas repression of ZEB1 signature and upregulation of MYC signature is associated with tumor-type agnostic resistance at Pan-cancer scale.

### Example 5: The iGenSig Model Predicted Patient Response to 5-FU Treatment in a French CIT Multicenter Study

Next, the utility of iGenSig modeling on predicting chemotherapy response was examined. While most clinical studies of chemo-agents focus on testing combination regimens, a multi-center clinical study carried out by the French Cartes d'dentité des Tumeurs (CIT) program was identified that tested 5-fluorouracil (5-FU) monotherapy on postsurgical colon cancer patients²³. In addition, this study also tested combination chemotherapy regimen such as FOLFIR1, FOLFOX, and FUFOL. 5-FU is an antimetabolite drug, and is one of the most commonly used drugs for cancer treatment, particularly for colorectal cancer²⁴. The GDSC iGenSig model significantly predicted patient overall survival in the 5-FU monotherapy arm (p=0.002), with a hazard ratio of 0.27 (FIG. 5A). This predictive effect was diminished in the treatment arms testing combination chemotherapies containing 5-FU (FIG. 5B). To examine if this is due to the therapeutic effect exerted by other chemo-agents, the FOLFIRI arm testing the combination of folinic acid, 5-FU, and irinotecan was examined, as the iGenSig models for the latter two drug are available. Among the alive patients in this arm, two patients showed high iGenSig scores by both models, whereas the other three patients showed high scores by either of these two models (FIG. 5C). This suggests that in the two alive patients with low iGenSig score by the 5-FU model, the therapeutic effects may be derived from irinotecan.

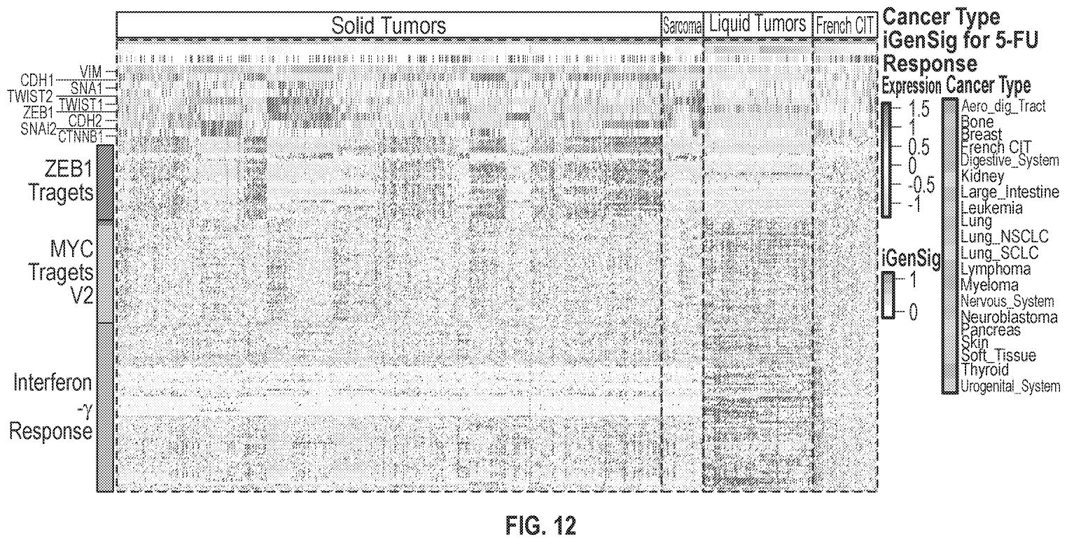

Next, the pathways enriched in the 5-FU sensitive iGenSig signature were examined. Interestingly, as opposed to the Erlotinib signature, the EMT pathway is ranked as the top downregulated pathway in the sensitive GDSC cell lines, whereas the MYC target gene signature and interferon γ signature are revealed as top upregulated pathways associated with sensitive responses. This suggests that the tumors that are resistant to EGFR inhibitors may be sensitive to the 5-FU treatment. Consistent with this, it is known that EGFR wild-type tumors show higher sensitivity to uracil-tegafur than EGFR mutated tumors in lung cancer²⁵, whereas EGFR inhibition has been found to sensitize 5-Fu-resistant colon cancer cells²⁶. Interferon γ signature is associated with inflammatory response triggered by the double strand breaks resulting from the DNA damaging effect of 5-FU. The upregulation of interferon γ regulated genes in cancer cells may confer better therapeutic effects through the interferon γ induced growth arrest and apoptosis in cancer cells^{27, 28}, and this signature appears to be captured from the leukemia and lymphoma cell lines in the GDSC panel (FIG. 12).

### Example 6: Applying iGenSig Models to a Clinical Trial of Combination Chemotherapy Provided Insights into the Confounding Factors of the Therapeutic Effects

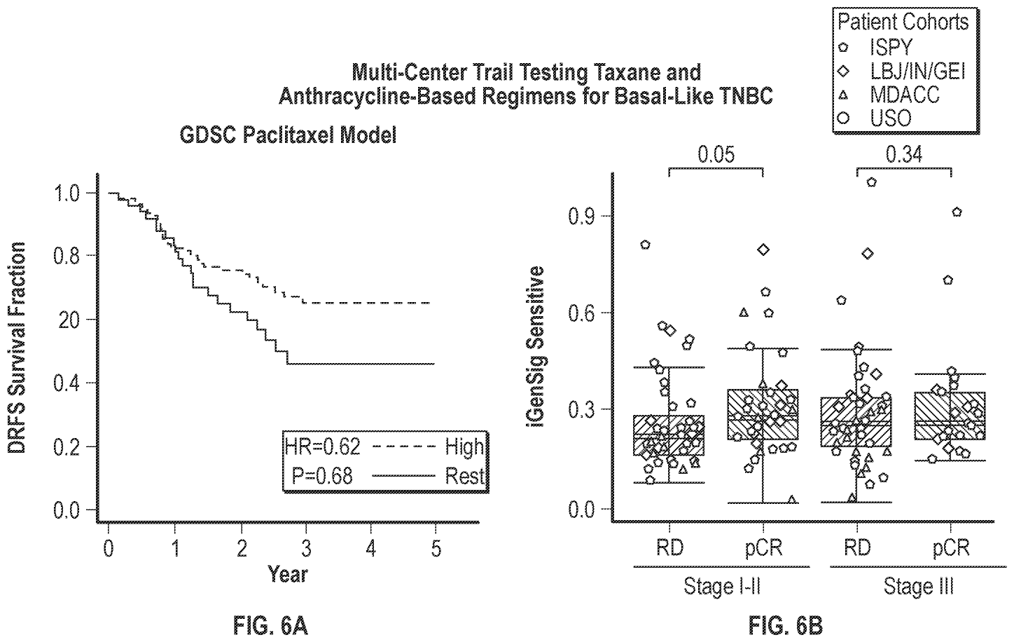

The predictive values of GDSC iGenSig models on clinical trials testing the combinatory chemotherapy regimens was further explored. To achieve this, a large gene expression dataset was identified from breast cancer clinical studies testing the drug combinations containing paclitaxel, for which favorable GDSC iGenSig model is obtained. The predictive effect of the paclitaxel model on a prospective clinical study for stage I-Ill breast cancer patient cohort treated with chemotherapy containing sequential taxane and anthracycline-based regimens was examined²¹. In this analysis, the basal-like triple negative breast cancer patients that are not treated with subsequent endocrine therapy were focused on. Results showed that the GDSC paclitaxel model showed moderate predictive effect on distant recurrence free survival, with a hazard ratio of 0.62 (FIG. 6A). When stratified by tumor stage, the predictive power of the model is more obviously in stage I-II tumors but is diminished in stage II tumors (FIG. 6B). Taken together, these data suggest that in combination therapy, the iGenSig models derived from single drug treatment data can be confounded by the therapeutic effect derived from other drugs in the combination, possible effects resulting from drug interactions, as well as the presentence of known confounding factors, such as tumor stage and ER positivity. Stratifying the patients based on known confounding factors will help better observe the predictive effect of the iGenSig models on combination drug therapies.

### Example 7: Comparison of the iGenSig Algorithm with Machine Learning Algorithms on Modeling Drug Responses

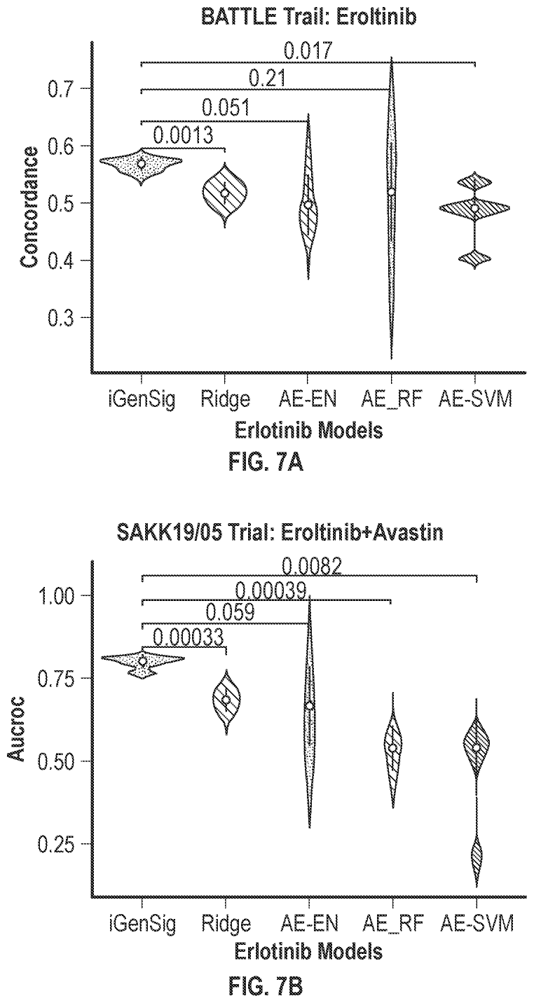

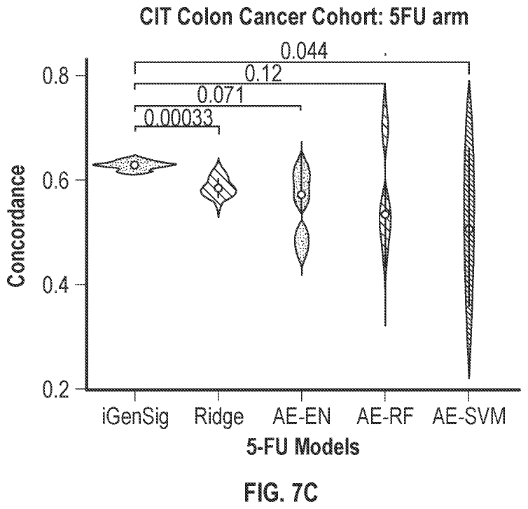

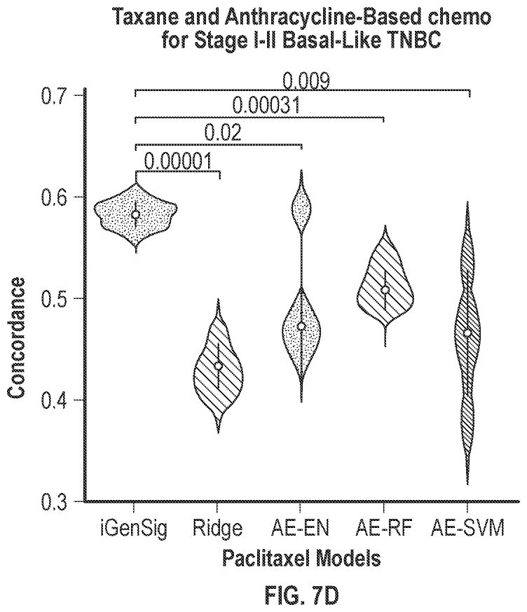

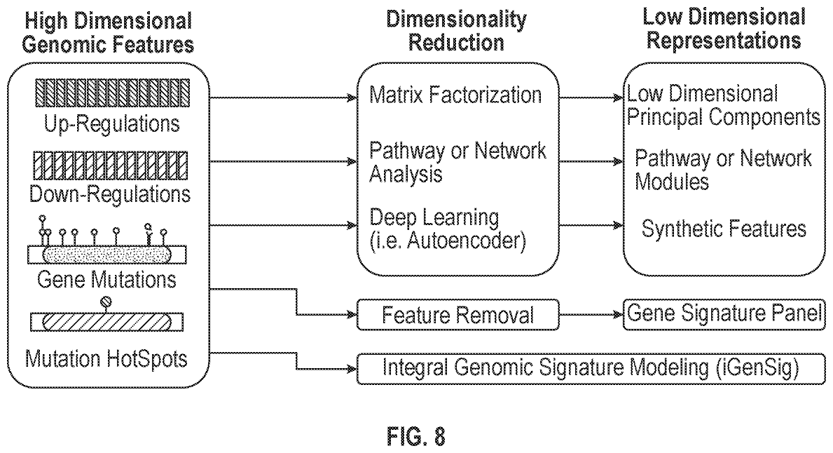

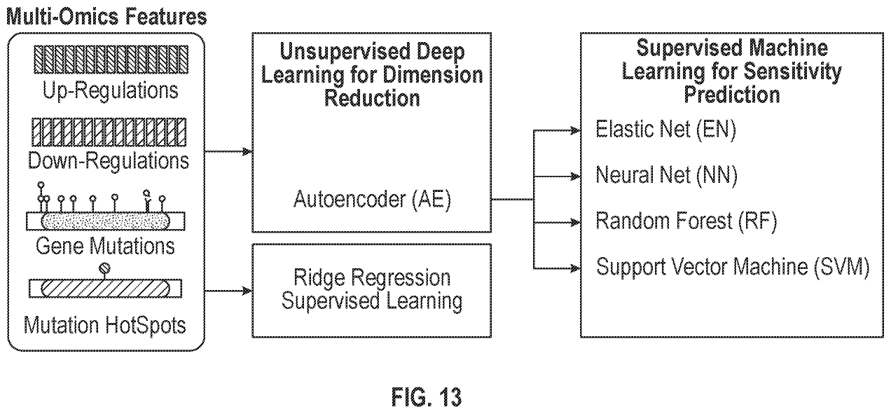

Next, the performance of the iGenSig algorithm was compared with machine learning or deep learning-based algorithms. For dimensionality reduction the unsupervised representation of the genomic features was computed based on the autoencoder deep learning method which were then fed to the machine learning methods for supervised learning on drug responses, such as elastic net, support vector machine (SVM) or Random Forest (RF)(FIG. 13). In addition to AI-based methods, iGenSig was compared with ridge regression, one of the few high-dimensional machine learning algorithms capable of carrying out predictive modeling without any dimension reduction using ultra-high dimensional features. These methods were then applied to model cancer cell sensitivity to the clinical trial datasets, including BATTLE, SAKK 19/05, and French CIT trials and the neoadjuvant taxane-anthracycline study. The results showed that the iGenSig models showed significantly better performance comparing to ridge regression and all other AI methods on predicting patient responses to the respective therapies in these clinical trials (FIG. 7A-D).

### Example 8: Clinical Applications of the iGenSig Approach

Provided herein is a new class of integral genomic signature methods that leverage the high-dimensional redundant genomic features as an integral genomic signature to enhance the resilience of multi-omics-based modeling for precision modeling, a concept like the use of redundant steel rods to reinforce the pillars of a building. The iGenSig method is designed to address the transparency, resilience, cross-dataset applicability, and interpretability issues for big-data based modeling. The iGenSig models demonstrated outstanding performances in cross-applicability to clinical trial datasets, tolerating the experimental variations and bias in the genomic data. iGenSig models can be managed in every detailed step, and the underlying pathways can be readily biologically interpreted through the concept signature enrichment analysis developed herewith. Without wishing to be bound by theory, the performance of iGenSig models appears to at least in part depend on the availability of significant genomic correlates which provided the insights into the different performances of iGenSig models on different drugs. iGenSig as a new class of big-data based modeling methods will have broad application in modeling therapeutic responses based on pharmacogenomic and clinical trial datasets.

### BIBLIOGRAPHY

1. Hilsenbeck, S. G. & Clark, G. M. Practical p-value adjustment for optimally selected cutpoints. Statistics in medicine 15, 103-112 (1996).
2. Langfelder, P., Zhang, B. & Horvath, S. Defining clusters from a hierarchical cluster tree: the Dynamic Tree Cut package for R. Bioinformatics 24, 719-720 (2008).
3. Irizarry, R. A. et al. Exploration, normalization, and summaries of high density oligonucleotide array probe level data. Biostatistics 4, 249-264 (2003).
4. Gao, H. et al. High-throughput screening using patient-derived tumor xenografs to predict clinical trial drug response. Nature medicine 21, 1318-1325 (2015).
5. Barretina, J. et al. The Cancer Cell Line Encyclopedia enables predictive modelling of anticancer drug sensitivity. Nature 483, 603-607 (2012).
6. Iorio, F. et al. A landscape of pharmacogenomic interactions in cancer. Cell 166, 740-754 (2016).
7. Subramanian, A. et al. Gene set enrichment analysis: a knowledge-based approach for interpreting genome-wide expression profiles. Proc Natl Acad Sci USA 102, 15545-15550(2005).
8. Driscoll, J. J. & Rixe, O. Overall survival: still the gold standard: why overall survival remains the definitive end point in cancer clinical trials. Cancer J 15, 401-405 (2009).
9. Gulli, A. & Pal, S. Deep learning with Keras. (Packt Publishing Ltd, 2017).
10. Ding, M. Q., Chen, L., Cooper. G. F., Young, J. D. & Lu, X. Precision Oncology beyond Targeted Therapy: Combining Omics Data with Machine Learning Matches the Majority of Cancer Cells to Effective Therapeutics. Mol Cancer Res 16, 269-278 (2018).
11. Friedman, J., Hastie, T. & Tibshirani, R. Regularization paths for generalized linear models via coordinate descent. Journal of statistical software 33, 1 (2010).
12. Chi, X. et al. Universal concept signature analysis: genome-wide quantification of new biological and pathological functions of genes and pathways. Brief Bioinform (2019).
13. Donato, M. et al. Analysis and correction of crosstalk effects in pathway analysis. Genome Res 23, 1885-1893 (2013).
14. Yu, L., Zhou, D., Gao, L. & Zha, Y. Prediction of drug response in multilayer networks based on fusion of multiomics data. Methods (2020).
15. Jang, I. S., Neto, E. C., Guinney, J., Friend, S. H. & Margolin, A. A. Systematic assessment of analytical methods for drug sensitivity prediction from cancer cell line data. Pac Symp Biocomput, 63-74 (2014).
16. Liberzon, A. A description of the Molecular Signatures Database (MSigDB) Web site. Methods Mol Biol 1150, 153-160 (2014).
17. Safikhani, Z. et al. Revisiting inconsistency in large pharmacogenomic studies. F1000Res 5, 2333 (2016).
18. Kim, E. S. et al. The BATTLE trial: personalizing therapy for lung cancer. Cancer Discov 1, 44-53 (2011).
19. Baty, F. et al. EGFR exon-level biomarkers of the response to bevacizumab/erlotinib in non-small cell lung cancer. PLoS One 8, e72966 (2013).
20. Byers, L. A. et al. An epithelial-mesenchymal transition gene signature predicts resistance to EGFR and PI3K inhibitors and identifies Axl as a therapeutic target for overcoming EGFR inhibitor resistance. Clin Cancer Res 19, 279-290 (2013).
21. Zhu, L. et al. Targeting c-Myc to Overcome Acquired Resistance of EGFR Mutant NSCLC Cells to the Third-Generation EGFR Tyrosine Kinase Inhibitor, Osimertinib. Cancer Res 81, 4822-4834(2021).
22. Taube, J. H. et al. Core epithelial-to-mesenchymal transition interactome gene-expression signature is associated with claudin-low and metaplastic breast cancer subtypes. Proc. Natl Acad Sci USA 107, 15449-15454 (2010).
23. Marisa, L. et al. Gene expression classification of colon cancer into molecular subtypes: characterization, validation, and prognostic value. PLoS Med 10, e1001453 (2013).
24. Longley, D. B., Harkin, D. P. & Johnston, P. G. 5-fluorouracil: mechanisms of action and clinical strategies. Nat Rev Cancer 3, 330-338 (2003).
25. Suehisa, H. et al. Epidermal growth factor receptor mutation status and adjuvant chemotherapy with uracil-tegafur for adenocarcinoma of the lung. J Clin Oncol 25, 3952-3957 (2007).
26. Gao, S. J., Ren, S. N., Liu, Y. T., Yan, H. W.& Chen, X. B. Targeting EGFR sensitizes 5-Fu-resistant colon cancer cells through modification of the lncRNA-FGDS-AS1-miR-330-3p-Hexokinase 2 axis. Mol Ther Oncolytics 23, 14-25 (2021).
27. Kotredes, K. P. & Gamero, A. M. Interferons as inducers of apoptosis in malignant cells. J Interferon Cytokine Res 33, 162-170 (2013).
28. Jorgovanovic, D., Song, M., Wang, L. & Zhang, Y. Roles of IFN-gamma in tumor progression and regression: a review. Biomark Res 8, 49 (2020).
29. Hatzis, C. et al. A genomic predictor of response and survival following taxane-anthracycline chemotherapy for invasive breast cancer. JAMA 305, 1873-1881 (2011).
30. Sharifi-Noghabi, H., Zolotareva, O., Collins, C. C. & Ester, M. MOLI: multi-omics late integration with deep neural networks for drug response prediction. Bioinformatics 35, i501-i509 (2019).

All publications and patents referred to herein are incorporated by reference. Various modifications and variations of the described subject matter will be apparent to those skilled in the art without departing from the scope and spirit of the invention. Although the invention has been described in connection with specific embodiments, it should be understood that the invention as claimed should not be unduly limited to these embodiments. Indeed, various modifications for carrying out the invention are obvious to those skilled in the art and are intended to be within the scope of the following claims.

What is claimed:

1-37. (canceled)

38. A method of modelling a therapeutic response of a cancer cell or tumor, comprising:
calculating a weight for each of a plurality of redundant multi-omics features that predict agent sensitivity or other clinical features based on statistical or machine learning methods; and
calculating a genomic signature score for the cancer cell or tumor based on the weights.

39. The method according to claim 38, including reducing the effect of feature redundancy via adaptively penalizing the redundant features detected in specific samples based on co-occurrence assessed using large cohorts of human cancer cells, cell lines, or tumors.

40. The method according to claim 38, wherein the genomic signature score of a given cancer cell or tumor is calculated using the below formula or its modifications:
$${GenSig}_{\vert Cell {Line}_{x}} = \frac{{\sum }_{i = 1}^{n} {EW}_{i}}{{EFN}_{i}} = \frac{{\sum }_{i = 1}^{n} {I}_{\{ i ∈ x \}} \frac{{ω}_{i}}{{ε}_{i}}}{\frac{n}{{\overline{ε}}_{T}}}$$
wherein ε is a penalization factor, ω is a weight, and EW is Effective Weight.

41. The method according to claim 38, wherein the weights are calculated based on weighted Kolmogorov-Smirnov (K-S) tests of Act Area or Area Under the Curve (AUC).

42. The method according to claim 38, wherein the method is implemented by a computer.

43. An iGenSig model for an agent that calculates the probability of response of a patient having a cancer or tumor to treatment with the agent, wherein the model is generated according to the method of claim 38.

44. The iGenSig model according to claim 43, wherein the agent is an EGFR inhibitor, a HER2 inhibitor, a CDK 4/6 inhibitor, a HDAC inhibitor, a BCL2 inhibitor, a JAK inhibitor, a PARP inhibitor, a ERK inhibitor, a MEK inhibitor, a BRAF inhibitor, irinotecan, topotecan, paclitaxel, 5-FU, Vincristine, Venetoclax, Epirubicin, or combinations thereof.

45. The iGenSig model according to claim 44, wherein the EGFR inhibitor is erlotinib, lapatinib, Afatinib, AZD3759.

46. The iGenSig model according to claim 44, wherein the BRAF inhibitor is encorafenib, vemurafenib, or dabrafenib.

47. The iGenSig model according to claim 44, wherein the MEK inhibitor is binimetinib, cobimetinib, selumetinib, or trametinib.

48. The iGenSig model according to claim 44, wherein the HER2 inhibitor is neratinib, trastuzumab, dacomitinib, lapatinib, tucatinib, or pertuzumab.

49. The iGenSig model according to claim 44, wherein the CDK4/6 inhibitor is Ribociclib.

50. The iGenSig model according to claim 44, wherein the HDAC inhibitor is CAY10603, or AR-42.

51. The iGenSig model according to claim 44, wherein the BCL2 inhibitor is Venetoclax.

52. The iGenSig model according to claim 44, wherein the PARP inhibitor is Niraparib.

53. The iGenSig model according to claim 44, wherein the ERK inhibitor is ERK_6604.

54. The iGenSig model according to claim 44, wherein the JAK inhibitor is AZ960.

55. A method for selecting a patient having a cancer or tumor for treatment with an agent, said method comprising:
employing an iGenSig model for the agent to calculate the probability of response of the patient to treatment with the agent; and
selecting the patient for treatment with the agent if the probability of response is above a chosen threshold of sensitive iGenSig score; and/or de-implementing the treatment with the agent to a patient if the probability of resistance is above a chosen threshold of resistant iGenSig score.

56. The method according to claim 55, wherein at least one step of the method is implemented by a computer.

57. The method according to claim 55, further comprising administering the agent to the patient.

58. The method according to claim 57, wherein an effective amount of the agent is administered to the patient.

59. The method according to claim 55, wherein the agent comprises a pharmaceutically acceptable carrier.

60. The method according to claim 55, wherein the chosen threshold is a probability of 50-95% predicted response rate, or 50-95% predicted resistance rate.

61. The method according to claim 55, wherein the agent is an EGFR inhibitor, a HER2 inhibitor, a CDK 4/6 inhibitor, a HDAC inhibitor, a BCL2 inhibitor, a JAK inhibitor, a PARP inhibitor, a ERK inhibitor, a MEK inhibitor, a BRAF inhibitor, irinotecan, topotecan, paclitaxel, 5-FU, Vincristine, Venetoclax, Epirubicin, or combinations thereof.

62. A method for selecting a patient having a cancer or tumor for treatment with an agent, said method comprising:
employing an iGenSig model for the agent to calculate the probability of response of the patient to treatment with the agent; and
selecting the patient for treatment with the agent if the probability of response is above a chosen threshold of sensitive iGenSig score; and/or de-implementing the treatment with the agent to a patient if the probability of resistance is above a chosen threshold of resistant iGenSig score, wherein the iGenSig model is generated according to the method of claim 38.

63. The method according to claim 62, wherein the agent is an EGFR inhibitor, a HER2 inhibitor, a CDK 4/6 inhibitor, a HDAC inhibitor, a BCL2 inhibitor, a JAK inhibitor, a PARP inhibitor, a ERK inhibitor, a MEK inhibitor, a BRAF inhibitor, irinotecan, topotecan, paclitaxel, 5-FU, Vincristine, Venetoclax, Epirubicin, or combinations thereof.
